(19) United States
(12) Patent Application Publication
(10) Pub. No.: US 2016/0303184 A1
(43) Pub. Date: Oct. 20, 2016

(54) VANCOMYCIN-SUGAR CONJUGATES AND USES THEREOF

(71) Applicant: JAWAHARLAL NEHRU CENTRE FOR ADVANCED SCIENTIFIC RESEARCH (JNCASR), Bangalore (IN)

(72) Inventors: Jayanta HALDAR, Bangalore (IN); Venkateswarlu YARLAGADDA, Bangalore (IN); Goutham Belagula MANJUNATH, Bangalore (IN); Mohini Mohan KONAI, Bangalore (IN)

(21) Appl. No.: 15/024,242

(22) PCT Filed: Sep. 16, 2014
(86) PCT No.: PCT/IB2014/001835

§ 371 Date: May 18, 2016

(30) Foreign Application Priority Data
Sep. 23, 2013 (IN) 4314/CHE/2013

(51) Int. Cl. A61K 38/12; A01N 43/90; A61K 47/48

(52) U.S. Cl. CPC A61K 38/12; A61K 47/48092; A01N 43/90

(57) ABSTRACT

The present disclosure relates to vancomycin-sugar conjugates, its stereoisomers, prodrugs and pharmaceutically acceptable salts thereof. The present disclosure also relates to process of preparation of the vancomycin-sugar conjugates, its stereoisomers, prodrugs, pharmaceutically acceptable salts thereof, and to pharmaceutical compositions containing them. The compounds of the present disclosure are useful in the treatment, prevention or suppression of diseases mediated by bacteria.

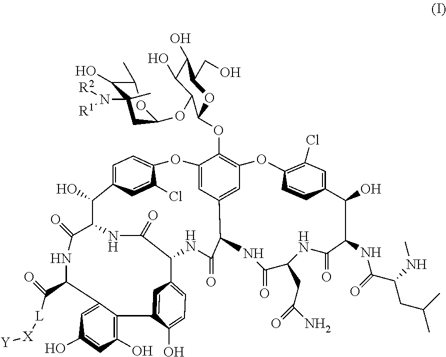

### FIELD OF INVENTION

The present disclosure relates to vancomycin-sugar conjugates, its stereoisomers, prodrugs and pharmaceutically acceptable salts thereof. The present disclosure further relates to a process of preparing the vancomycin-sugar conjugates, its stereoisomers, prodrugs and pharmaceutically acceptable salts thereof. The present disclosure also relates to compositions and methods of treating conditions and diseases that are mediated by bacteria.

### BACKGROUND

Vancomycin is a complex multi-ring glycopeptide and considered to be the drug of last resort for gram positive bacteria induced infections. It is effective as an antibacterial agent against a majority of gram-positive bacteria because of its unusual mode of action.

In its mechanism of action, vancomycin inhibits bacterial cell wall synthesis by binding to the peptidoglycan peptide terminus D-Ala-D-Ala found in the bacterial cell wall precursors, sequestering the substrate from transpeptidase and inhibiting cell wall cross-linking. However, some virulent bacterial species, such as vancomycin resistant S. aureus (VRSA) and vancomycin-resistant Enterococci (VRE), have acquired resistance to vancomycin by modifying their peptidoglycan terminus, changing from D-Ala-D-Ala to D-AlaD-Lac and/or thickening their cell wall. In the present scenario, curing of these drug resistant infections is deeply restricted by the scarcity of effective antibiotics. Significant efforts have been directed toward the discovery of next-generation glycopeptide antibiotics that address the emerging drug-resistance of bacteria, especially vancomycin resistant strains.

U.S. Pat. No. 4,639,433, U.S. Pat. No. 4,643,987, U.S. Pat. No. 4,497,802, U.S. Pat. No. 4,698,327, U.S. Pat. No. 5,591,714, U.S. Pat. No. 5,840,684 and U.S. Pat. No. 5,843,889 discloses derivatives of vancomycin and other derivatives.

U.S. Pat. No. 5,919,756 discloses glycopeptide amides which are useful for the control of gram positive bacteria, particularly useful for the control of resistant bacterial strains, such as VRE.

U.S. Pat. No. 8,030,445 discloses a novel derivative of glycopeptide antibiotics. The glycopeptide antibiotics are useful as antibacterial agents.

U.S. Pat. No. 6,444,786 discloses derivatives of glycopeptide compounds having at least one substituent, and pharmaceutical compositions containing such glycopeptide derivatives.

WO2001098327 discloses a saccharide derivative of glycopeptide antibiotics and related compounds having highly effective antibacterial activity.

WO2000042067 relates to saccharide compounds having transglycosylase inhibitory activity linked to non-saccharide compounds that bind to molecules located at the bacterial cell surface.

From the foregoing it is clear that compounds used in the state of the art to treat and prevent bacterial infection have been found to have limited effect against certain bacterial infections caused by glycopeptide resistant Enterococci. Further, there is a continuing need to identify new compounds which possess improved antibacterial activity, which have less potential for developing resistance, which possess improved effectiveness against bacterial infections that resist treatment with currently available antibiotics, or which possess unexpected selectivity against target microorganisms.

A need exists, however, for glycopeptide derivatives having improved activity, selectivity and reduced mammalian toxicity.

### SUMMARY

The present disclosure provides a compound of formula I

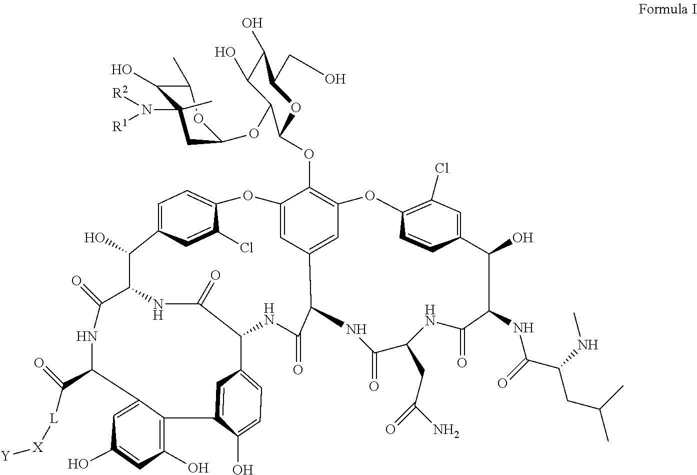

or its stereoisomers, prodrugs and pharmaceutically acceptable salts thereof:
wherein
R¹ and R² are independently selected from the group consisting of hydrogen, a C₂-C₁₈ alkyl, a C₆-C₁₈ aryl, alkenyl, alkynyl, haloalkyl, arylalkyl, hydroxyalkyl, carboxyalkyl, cycloalkyl, cycloalkylalkyl, heterocyclyl, heterocyclylalkyl, heteroaryl; wherein alkyl, alkenyl, alkynyl, cycloalkyl, cycloalkylalkyl, arylalkyl, aryl, heteroaryl, heterocyclyl, and heterocyclylalkyl are independently unsubstituted or substituted with up to four substituents independently selected from alkyl, alkenyl, alkynyl, alkoxy, acyl, acyloxy, acylamino, amino, monoalkylamino, dialkylamino, trialkylamino, halogen, hydroxy, hydroxyalkyl, keto, thiocarbonyl, carboxy, alkylcarboxy, hydroxyamino, alkoxyamino, nitro, azido, cyano, cycloalkyl, cycloalkylalkyl, aryl, arylalkyl, heterocyclyl, heterocyclylalkyl, heteroaryl, heteroarylalkyl, cycloalkenyl, cycloalkylamino, arylamino, heterocyclylamino, heteroarylamino, cycloalkyloxy, aryloxy, heterocyclyloxy or heteroaryloxy;
L is a C₂-C₆ alkyl, a C₈-C₁₈ aryl, alkenyl, alkynyl, haloalkyl, arylalkyl, hydroxyalkyl, carboxyalkyl, cycloalkyl, cycloalkylalkyl, heterocyclyl, heterocyclylalkyl, heteroaryl; wherein alkyl, alkenyl, alkynyl, cycloalkyl, cycloalkylalkyl, arylalkyl, aryl, heteroaryl, heterocyclyl, and heterocyclylalkyl are independently unsubstituted or substituted with upto four substituents independently selected from alkyl, alkenyl, alkynyl, alkoxy, acyl, acyloxy, acylamino, amino, halogen, hydroxy, hydroxyalkyl, keto, thiocarbonyl, carboxy, alkylcarboxy, hydroxyamino, alkoxyamino, nitro, azido, cyano, cycloalkyl, cycloalkylalkyl, aryl, arylalkyl, heterocyclyl, heterocyclylalkyl, heteroaryl, heteroarylalkyl, cycloalkenyl, cycloalkylamino, arylamino, heterocyclylamino, heteroarylamino, cycloalkyloxy, aryloxy, heterocyclyloxy or heteroaryloxy;

### X is NH and O; and

Y is selected from the group consisting of cyclic monosaccharide, cyclic disaccharide, acyclic monosaccharide, acyclic disaccharide, and combinations thereof.

The present disclosure further relates to a compound of formula I or its stereoisomers, prodrugs and pharmaceutically acceptable salts thereof, for use as a medicament.

The present disclosure relates to a pharmaceutical composition comprising a compound of formula I or its stereoisomers, prodrugs and pharmaceutically acceptable salts thereof, together with a pharmaceutically acceptable carrier.

The present disclosure relates to a process for preparation of compound of formula I or its stereoisomers, prodrugs and pharmaceutically acceptable salts thereof.

These and other features, aspects, and advantages of the present subject matter will become better understood with reference to the following description. This summary is provided to introduce a selection of concepts in a simplified form. This summary is not intended to identify key features or essential features of the disclosure, nor is it intended to be used to limit the scope of the subject matter.

### BRIEF DESCRIPTION OF DRAWINGS

The detailed description is described with reference to the accompanying figures. In the figures, the left-most digit(s) of a reference number identifies the figure in which the reference number first appears. The same numbers are used throughout the drawings to reference like features and components.

FIG. 1 illustrates ex-vivo whole blood assay of vancomycin-sugar conjugate.

FIG. 2 illustrates in-vivo time dependent whole blood assay of vancomycin-sugar conjugate.

FIG. 3 illustrates in-vitro time time-kill kinetics of vancomycin-sugar conjugate. The points below the dotted line in the figure indicates >3 log₁₀ CFU/mL reduction.

FIG. 4A illustrates experimental design of in-vivo activity of compound 7 in comparison with vancomycin and linezolid against MR-VISA.

FIG. 4B illustrates in-vivo activity of compound 7 in comparison with vancomycin and linezolid against MR-VISA.

FIG. 5A illustrates experimental design of pharmacodynamics of compound 7 in comparison against MR-VISA.

FIG. 5B illustrates pharmacodynamics of compound 7 in comparison against MR-VISA.

FIG. 6A illustrates experimental design of single-dose concentration-versus-time pharmacokinetic profile of compound 7 at 12 mg/kg.

FIG. 6B illustrates single-dose concentration-versus-time pharmacokinetic profile of compound 7 at 12 mg/kg.

### DETAILED DESCRIPTION

In the structural formulae given herein and throughout the present disclosure, the following terms have been indicated meaning, unless specifically stated otherwise.

### DEFINITIONS

The term “alkyl” refers to a monoradical branched or unbranched saturated hydrocarbon chain having from 1 to 18 carbon atoms, more preferably 1 to 12 carbon atoms. This term is exemplified by groups such as methyl, ethyl, n-propyl, iso-propyl, n-butyl, iso-butyl, t-butyl, n-hexyl, n-decyl, tetradecyl, and the like.

The term “substituted alkyl” refers to an alkyl group as defined above, having 1, 2, 3, or 4 substituents, preferably 1, 2 or 3 substituents, selected from the group consisting of alkyl, alkenyl, alkynyl, alkoxy, acyl, acyloxy, acylamino, amino, monoalkylamino, dialkylamino, trialkylamino, halogen, hydroxy, hydroxyalkyl, keto, thiocarbonyl, carboxy, alkylcarboxy, hydroxyamino, alkoxyamino, nitro, azido, cyano, cycloalkyl, cycloalkylalkyl, aryl, arylalkyl, heterocyclyl, heterocyclylalkyl, heteroaryl, heteroarylalkyl, cycloalkenyl, cycloalkylamino, arylamino, heterocyclylamino, heteroarylamino, cycloalkyloxy, aryloxy, heterocyclyloxy or heteroaryloxy;

The term “alkenyl” refers to a monoradical of a branched or unbranched unsaturated hydrocarbon group preferably having from 2, 3, 4, 5, 6, 7, 8, 9, 10, 11, 12, 13, 14, 15, 16, 17, 18, 19 or 20 carbon atoms, more preferably 2, 3, 4, 5, 6, 7, 8, 9 or 10 carbon atoms and even more preferably 2, 3, 4, 5 or 6 carbon atoms and having 1, 2, 3, 4, 5 or 6 double bond (vinyl), preferably 1 double bond. Preferred alkenyl groups include ethenyl or vinyl (—CH═CH₂), 1-propylene or allyl (—CH₂CH═CH₂), isopropylene (—C(CH₃)═CH₂), bicyclo [2.2.1] heptene, and the like.

The term “substituted alkenyl” refers to an alkenyl group as defined above having 1, 2, 3, or 4 substituents, and preferably 1, 2, or 3 substituents, selected from the group consisting of alkyl, alkenyl, alkynyl, alkoxy, acyl, acyloxy, acylamino, amino, halogen, hydroxy, hydroxyalkyl, keto, thiocarbonyl, carboxy, alkylcarboxy, hydroxyamino, alkoxyamino, nitro, azido, cyano, cycloalkyl, cycloalkylalkyl, aryl, arylalkyl, heterocyclyl, heterocyclylalkyl, heteroaryl, heteroarylalkyl, cycloalkenyl, cycloalkylamino, arylamino, heterocyclylamino, heteroarylamino, cycloalkyloxy, aryloxy, heterocyclyloxy or heteroaryloxy.

The term “alkynyl” refers to a monoradical of an unsaturated hydrocarbon, preferably having from 2, 3, 4, 5, 6, 7, 8, 9, 10, 11, 12, 13, 14, 15, 16, 17, 18, 19 or 20 carbon atoms, more preferably 2, 3, 4, 5, 6, 7, 8, 9 or 10 carbon atoms and even more preferably 2, 3, 4, 5 or 6 carbon atoms and having 1, 2, 3, 4, 5 or 6 sites of acetylene (triple bond) unsaturation, preferably 1 triple bond. Preferred alkynyl groups include ethynyl, (—C≡CH), propargyl (or prop-1-yn-3-yl, —CH₂C≡CH), homopropargyl (or but-1-yn-4-yl, —CH₂CH₂C≡CH) and the like.

The term “substituted alkynyl” refers to an alkynyl group as defined above having 1, 2, 3, or 4 substituents, and preferably 1, 2, or 3 substituents, selected from the group consisting of alkyl, alkenyl, alkynyl, alkoxy, acyl, acyloxy, acylamino, amino, halogen, hydroxy, hydroxyalkyl, keto, thiocarbonyl, carboxy, alkylcarboxy, hydroxyamino, alkoxyamino, nitro, azido, cyano, cycloalkyl, cycloalkylalkyl, aryl, arylalkyl, heterocyclyl, heterocyclylalkyl, heteroaryl, heteroarylalkyl, cycloalkenyl, cycloalkylamino, arylamino, heterocyclylamino, heteroarylamino, cycloalkyloxy, aryloxy, heterocyclyloxy or heteroaryloxy;

“Halo” or “Halogen”, alone or in combination with any other term means halogens such as chloro (Cl), fluoro (F), bromo (Br) and iodo (I).

“Haloalkyl” refers to a straight chain or branched chain haloalkyl group with 1 to 6 carbon atoms. The alkyl group may be partly or totally halogenated. Representative examples of haloalkyl groups include but are not limited to fluoromethyl, chloromethyl, bromomethyl, difluoromethyl, dichloromethyl, dibromomethyl, trifluoromethyl, trichloromethyl, 2-fluoroethyl, 2-chloroethyl, 2-bromoethyl, 2,2,2-trifluoroethyl, 3-fluoropropyl, 3-chloropropyl, 3-bromopropyl and the like.

The term “aryl” refers to an aromatic carbocyclic group of 6 to 18 carbon atoms having a single ring (e.g. phenyl) or multiple rings (e.g. biphenyl), or multiple condensed (fused) rings (e.g. naphthyl or anthranyl). Preferred aryls include phenyl, naphthyl and the like.

The term “substituted aryl” refers to an alkynyl group as defined above having 1, 2, 3, or 4 substituents, and preferably 1, 2, or 3 substituents, selected from the group consisting of alkyl, alkenyl, alkynyl, alkoxy, acyl, acyloxy, acylamino, amino, halogen, hydroxy, hydroxyalkyl, keto, thiocarbonyl, carboxy, alkylcarboxy, hydroxyamino, alkoxyamino, nitro, azido, cyano, cycloalkyl, cycloalkylalkyl, aryl, arylalkyl, heterocyclyl, heterocyclylalkyl, heteroaryl, heteroarylalkyl, cycloalkenyl, cycloalkylamino, arylamino, heterocyclylamino, heteroarylamino, cycloalkyloxy, aryloxy, heterocyclyloxy or heteroaryloxy;

The term “arylalkyl” refers to an aryl group covalently linked to an alkylene group, where aryl and alkylene are defined herein.

The term “hydroxyalkyl” refers to the groups -alkylene-OH.

The term “carboxyalkyl” refers to the groups -alkylene-C(O)OH.

The term “cycloalkyl” refers to carbocyclic groups of from 3 to 20 carbon atoms having a single cyclic ring or multiple condensed rings which may be partially unsaturated. Such cycloalkyl groups include, by way of example, single ring structures such as cyclopropyl, cyclobutyl, cyclopentyl, cyclopentenyl, cyclohexyl, cyclohexenyl, cyclooctyl, and the like, or multiple ring structures such as adamantanyl, bicyclo[2.2.1]heptane, 1,3,3-trimethylbicyclo[2.2.1]hept-2-yl, (2,3,3-trimethylbicyclo[2.2.1]hept-2-yl), or carbocyclic groups to which is fused an aryl group, for example indane, and the like.

The term “substituted cycloalkyl” refers to cycloalkyl groups having 1, 2, 3, or 4 substituents, and preferably 1, 2, or 3 substituents, selected from the group consisting of alkyl, alkenyl, alkynyl, alkoxy, acyl, acyloxy, acylamino, amino, halogen, hydroxy, hydroxyalkyl, keto, thiocarbonyl, carboxy, alkylcarboxy, hydroxyamino, alkoxyamino, nitro, azido, cyano, cycloalkyl, cycloalkylalkyl, aryl, arylalkyl, heterocyclyl, heterocyclylalkyl, heteroaryl, heteroarylalkyl, cycloalkenyl, cycloalkylamino, arylamino, heterocyclylamino, heteroarylamino, cycloalkyloxy, aryloxy, heterocyclyloxy or heteroaryloxy;

“Cycloalkylalkyl” refers to an alkyl radical as defined above which is substituted by a cycloalkyl radical as defined above. Representative examples of cycloalkylalkyl include but are not limited to cyclopropylmethyl, cyclobutylmethyl, cyclopentylmethyl, cyclohexylmethyl, 1-cyclopentylethyl, 1-cyclohexylethyl, 2-cyclopentylethyl, 2-cyclohexylethyl, cyclobutylpropyl, cyclopentylpropyl, cyclohexylbutyl and the like.

The term “heterocyclyl” refers to a saturated or partially unsaturated group having a single ring or multiple condensed rings, having from 1 to 40 carbon atoms and from 1 to 10 hetero atoms, preferably 1, 2, 3 or 4 heteroatoms, selected from nitrogen, sulfur, phosphorus, and/or oxygen within the ring. Heterocyclic groups can have a single ring or multiple condensed rings, and include tetrahydrofuranyl, morpholinyl, piperidinyl, piperazinyl, dihydropyridinyl, tetrahydroquinolinyl, pyrrolidinyl and the like.

The term “heterocyclylalkyl” refers to a heterocyclyl group covalently linked to an alkylene group, where heterocyclyl and alkylene are defined herein.

The term “heteroaryl” refers to an aromatic cyclic group having 1, 2, 3, 4, 5, 6, 7, 8, 9, 10, 11, 12, 13, 14, or 15 carbon atoms and 1, 2, 3 or 4 heteroatoms selected from oxygen, nitrogen and sulfur within at least one ring (if there is more than one ring). Such heteroaryl groups can have a single ring (e.g. pyridyl or furyl) or multiple condensed rings (e.g. indolizinyl, benzothiazolyl, or benzothienyl). Examples of heteroaryls include, but are not limited to, [1,2,4] oxadiazole, [1,3,4] oxadiazole, [1,2,4] thiadiazole, [1,3,4] thiadiazole, pyrrole, imidazole, pyrazole, pyridine, pyrazine, pyrimidine, pyridazine, indolizine, isoindole, indole, indazole, purine, quinolizine, isoquinoline, quinoline, phthalazine, quinoxaline, quinazoline, cinnoline, pteridine, carbazole, carboline, phenanthridine, acridine, phenanthroline, isothiazole, phenazine, isoxazole, phenoxazine, phenothiazine, furan, thiophene, oxazole, thiazole, triazole, triazine and the like.

The compounds described herein may contain one or more chiral centers and/or double bonds and therefore, may exist as stereoisomers, such as double-bond isomers (i.e., geometric isomers), regioisomers, enantiomers or diastereomers. Accordingly, the chemical structures depicted herein encompass all possible enantiomers and stereoisomers of the illustrated or identified compounds including the stereoisomerically pure form (e.g., geometrically pure, enantiomerically pure or diastereomerically pure) and enantiomeric and stereoisomeric mixtures. Enantiomeric and stereoisomeric mixtures can be resolved into their component enantiomers or stereoisomers using separation techniques or chiral synthesis techniques well known to the person skilled in the art. The compounds, may also exist in several tautomeric forms including the enol form, the keto form and mixtures thereof. Accordingly, the chemical structures depicted herein encompass all possible tautomeric forms of the illustrated or identified compounds.

“Pharmaceutically acceptable salt” embraces salts with a pharmaceutically acceptable acid or base. Pharmaceutically acceptable acids include both inorganic acids, for example hydrochloric, sulphuric, phosphoric, diphosphoric, hydrobromic, hydroiodic and nitric acid and organic acids, for example citric, fumaric, maleic, malic, mandelic, ascorbic, oxalic, succinic, tartaric, benzoic, acetic, methanesulphonic, ethanesulphonic, benzenesulphonic or p-toluenesulphonic acid. Pharmaceutically acceptable bases include alkali metal (e.g. sodium or potassium) and alkali earth metal (e.g. calcium or magnesium) hydroxides and organic bases, for example alkyl amines, arylalkyl amines and heterocyclic amines.

“Glycopeptide” refers to a heptapeptide antibiotics characterized by a multi-ring peptide core substituted with a saccharide groups.

“Saccharide” refers to a simple sugar or a compound with sugars linked to each other. Saccharides are classified as mono-, di-, tri-, and polysaccharides according to the number of monosaccharide groups composing them.

The term “peptide” refers to a compound consisting of two or more amino acids linked in a chain, the carboxyl group of each acid being joined to the amino group

“Vancomycin” refers to the glycopeptide antibiotic having the structural formula

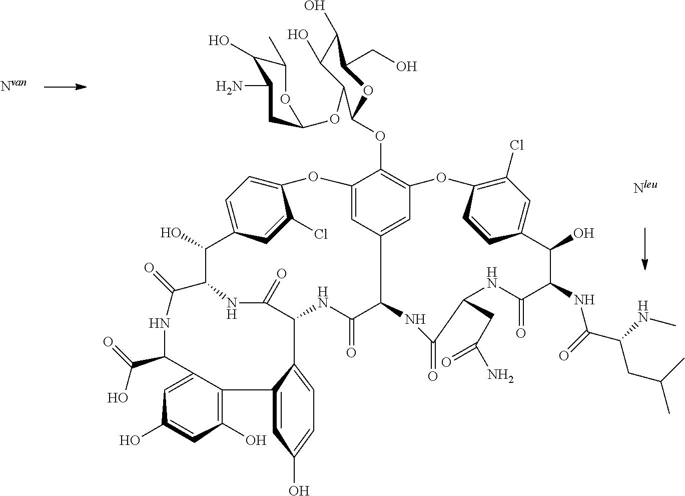

and is also represented in the disclosure by the formula provided below:

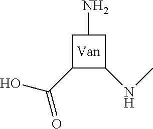

wherein —NH₂, —NHCH₃ represents N^{van}, and N^{leu} respectively.

Vancosamine moiety of vancomycin is shown as the N-site where a substituent can be covalently attached to the structure of Vancomycin.

The present disclosure provides a compound of formula I

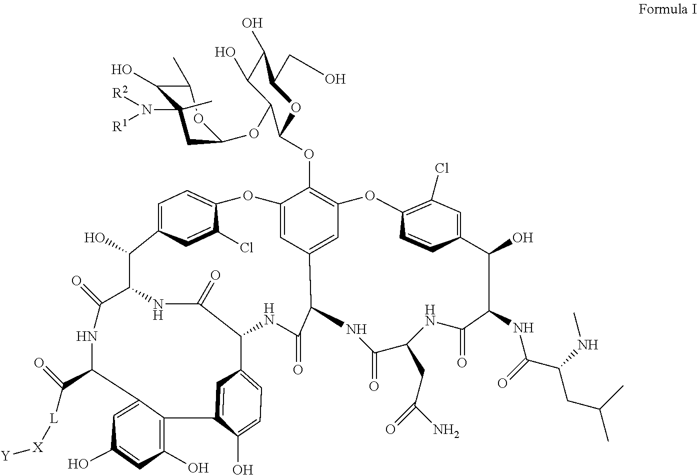

or its stereoisomers, prodrugs and pharmaceutically acceptable salts thereof:
wherein
R¹ and R² are independently selected from the group consisting of hydrogen, a C₂-C₁₈ alkyl, a C₆-C₁₈ aryl, alkenyl, alkynyl, haloalkyl, arylalkyl, hydroxyalkyl, carboxyalkyl, cycloalkyl, cycloalkylalkyl, heterocyclyl, heterocyclylalkyl, heteroaryl; wherein alkyl, alkenyl, alkynyl, cycloalkyl, cycloalkylalkyl, arylalkyl, aryl, heteroaryl, heterocyclyl, and heterocyclylalkyl are independently unsubstituted or substituted with upto four substituents independently selected from alkyl, alkenyl, alkynyl, alkoxy, acyl, acyloxy, acylamino, amino, monoalkylamino, dialkylamino, trialkylamino, halogen, hydroxy, hydroxyalkyl, keto, thiocarbonyl, carboxy, alkylcarboxy, hydroxyamino, alkoxyamino, nitro, azido, cyano, cycloalkyl, cycloalkylalkyl, aryl, arylalkyl, heterocyclyl, heterocyclylalkyl, heteroaryl, heteroarylalkyl, cycloalkenyl, cycloalkylamino, arylamino, heterocyclylamino, heteroarylamino, cycloalkyloxy, aryloxy, heterocyclyloxy or heteroaryloxy;
L is a C₂-C₆ alkyl, a C₈-C₁₈ aryl, alkenyl, alkynyl, haloalkyl, arylalkyl, hydroxyalkyl, carboxyalkyl, cycloalkyl, cycloalkylalkyl, heterocyclyl, heterocyclylalkyl, heteroaryl; wherein alkyl, alkenyl, alkynyl, cycloalkyl, cycloalkylalkyl, arylalkyl, aryl, heteroaryl, heterocyclyl, and heterocyclylalkyl are independently unsubstituted or substituted with upto four substituents independently selected from alkyl, alkenyl, alkynyl, alkoxy, acyl, acyloxy, acylamino, amino, halogen, hydroxy, hydroxyalkyl, keto, thiocarbonyl, carboxy, alkylcarboxy; hydroxyamino, alkoxyamino, nitro, azido, cyano, cycloalkyl, cycloalkylalkyl, aryl, arylalkyl, heterocyclyl, heterocyclylalkyl, heteroaryl, heteroarylalkyl, cycloalkenyl, cycloalkylamino, arylamino, heterocyclylamino, heteroarylamino, cycloalkyloxy, aryloxy, heterocyclyloxy or heteroaryloxy;

### X is NH, and O; and

Y is selected from the group consisting of cyclic monosaccharide, cyclic disaccharide, acyclic monosaccharide, acyclic disaccharide, and combinations thereof.

According to an embodiment, the present disclosure relates to compounds of formula I or its stereoisomers, prodrugs and pharmaceutically acceptable salts thereof:

wherein
R¹ is hydrogen;
R² is selected from the group consisting of hydrogen, a C₃-C₁₈ alkyl, and a C₆-C₁₈ aryl; wherein alkyl, aryl, are independently unsubstituted or substituted with two substituents independently selected from alkyl, halogen, hydroxy, monoalkylamino, dialkylamino, trialkylamino, nitro, aryl;
L is a C₂-C₆ alkyl;

### X is NH, and O; and

Y is selected from the group consisting of cyclic monosaccharide, cyclic disaccharide, acyclic monosaccharide, acyclic disaccharide, and combinations thereof.

According to an embodiment, the present disclosure relates to compounds of formula I or its stereoisomers, prodrugs and pharmaceutically acceptable salts thereof:

wherein
R¹ is hydrogen;
R² is selected from the group consisting of hydrogen, a C₂-C₁₂ alkyl; wherein alkyl is independently unsubstituted or substituted with two substituents independently selected from alkyl, halogen, hydroxy, monoalkylamino, dialkylamino, trialkylamino, nitro, aryl;
L is a C₂-C₆ alkyl;

### X is NH, and O; and

Y is selected from the group consisting of cyclic monosaccharide, cyclic disaccharide, acyclic monosaccharide, acyclic disaccharide, and combinations thereof.

According to another embodiment, the present disclosure relates to compounds of formula I or its stereoisomers, prodrugs and pharmaceutically acceptable salts thereof: wherein Y is selected from the group consisting of

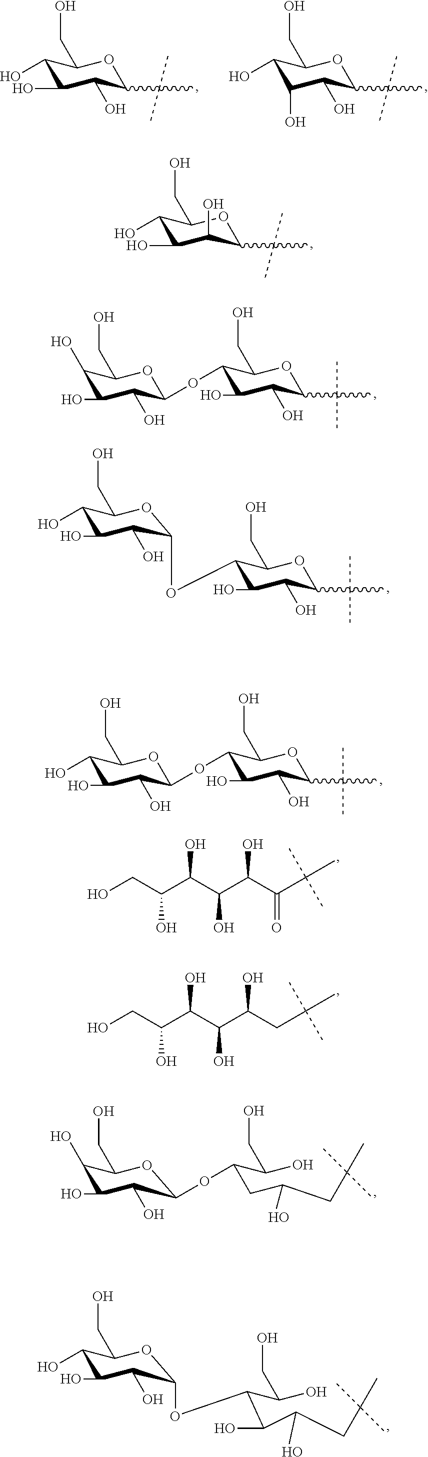

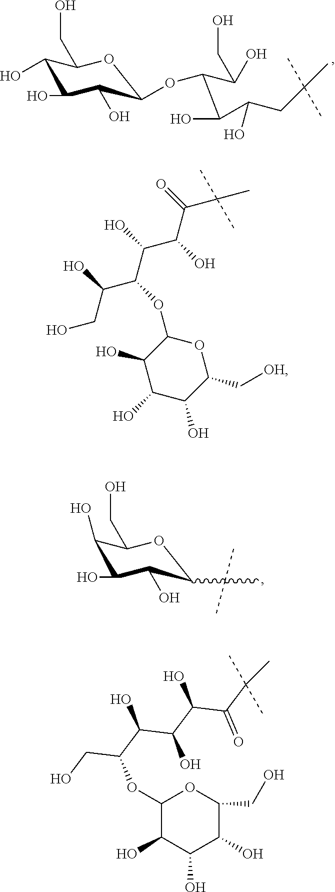

According to another embodiment, the present disclosure relates to compounds of formula I or its stereoisomers, prodrugs and pharmaceutically acceptable salts thereof: wherein Y is selected from the group consisting of

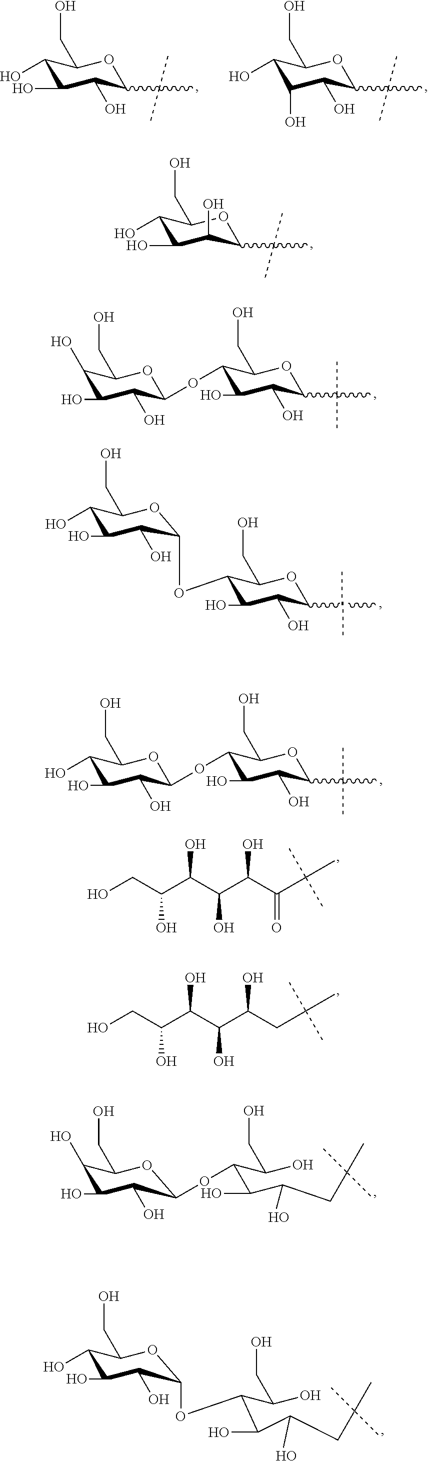

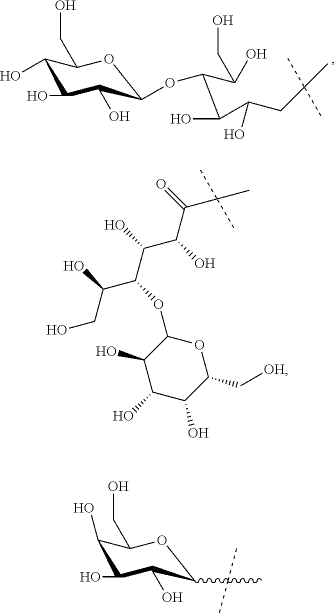

According to yet another embodiment, the present disclosure relates to compounds of formula I or its stereoisomers, prodrugs and pharmaceutically acceptable salts thereof:

wherein
R¹ is hydrogen;
R² is selected from the group consisting of hydrogen, a C₂-C₁₂ alkyl, and a C₆-C₁₈ aryl; wherein alkyl, aryl, are independently unsubstituted or substituted with two substituents independently selected from alkyl, halogen, hydroxy, monoalkylamino, dialkylamino, trialkylamino, nitro, aryl.
L is a C₂-C₆ alkyl;

### X is NH, and O; and

Y is selected from the group consisting of

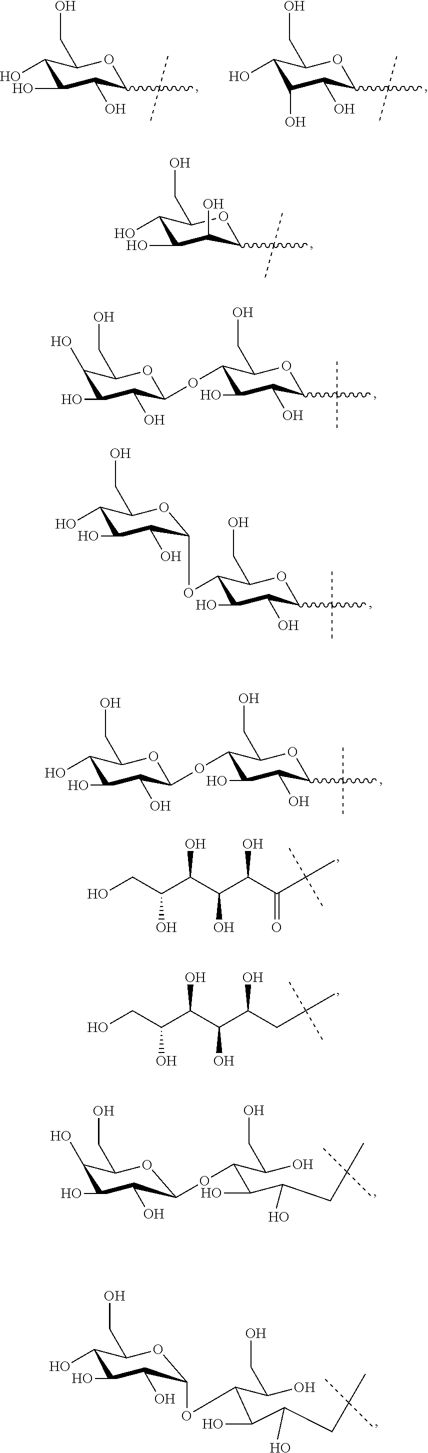

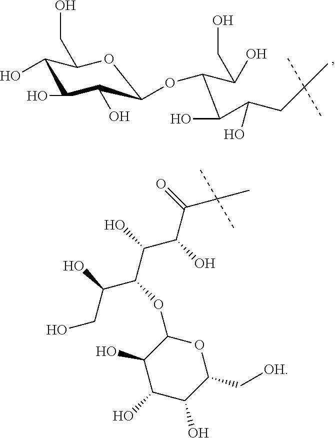

According to an embodiment, the present disclosure relates to compounds of formula I or its stereoisomers, prodrugs and pharmaceutically acceptable salts thereof:

wherein
R¹ is hydrogen;
R² is selected from the group consisting of hydrogen, and C₆-C₁₈ alkyl;
L is a C₂-C₆ alkyl;

### X is NH, and O; and

Y is selected from the group consisting of

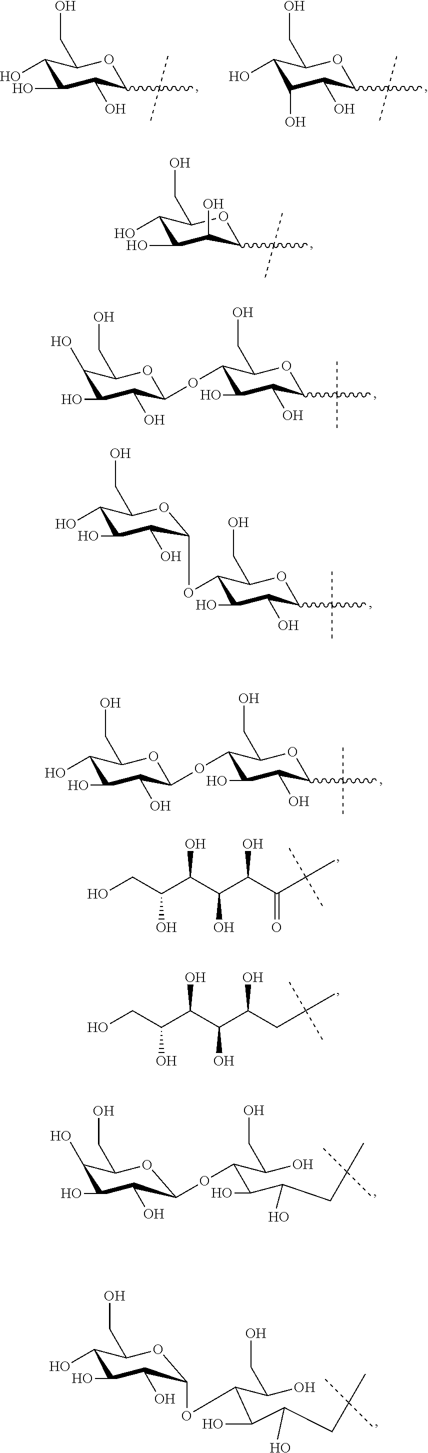

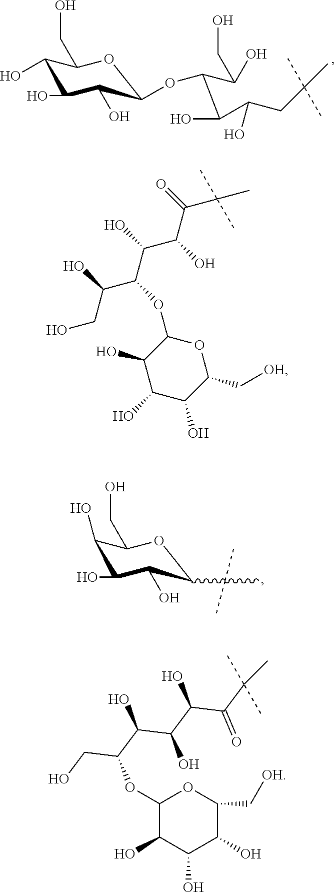

According to another embodiment, the present disclosure relates to compounds of formula I or its stereoisomers, prodrugs and pharmaceutically acceptable salts thereof:

wherein
R¹ is hydrogen;
R² is selected from the group consisting of hydrogen, a C₆-C₁₈ alkyl, and a C₆-C₁₈ aryl;
L is a C₂-C₆ alkyl;

### X is NH, and O; and

Y is selected from the group consisting of

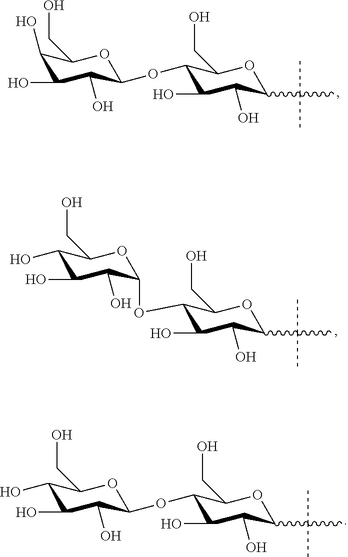

According to yet another embodiment, the present disclosure relates to compounds of formula I or its stereoisomers, prodrugs and pharmaceutically acceptable salts thereof:

wherein
R¹ is hydrogen;
R² is selected from the group consisting of hydrogen, a C₂-C₁₂ alkyl, and a C₆-C₁₈ aryl; wherein alkyl, aryl, are independently unsubstituted or substituted with two substituents independently selected from alkyl, halogen, hydroxy, monoalkylamino, dialkylamino, trialkylamino, nitro, and aryl.
L is a C₂-C₆ alkyl;

### X is NH, and O; and

Y is selected from the group consisting of

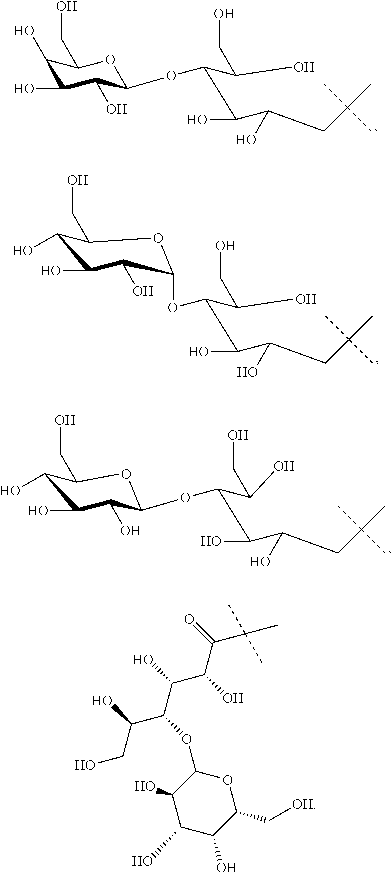

According to another embodiment, the present disclosure relates to compounds of formula I or its stereoisomers, prodrugs and pharmaceutically acceptable salts thereof:

wherein
R¹ is hydrogen;
R² selected from the group consisting of hydrogen, and a C₆-C₁₈ alkyl;
L is a C₂-C₆ alkyl;

### X is NH, or O;

Y is selected from the group consisting of

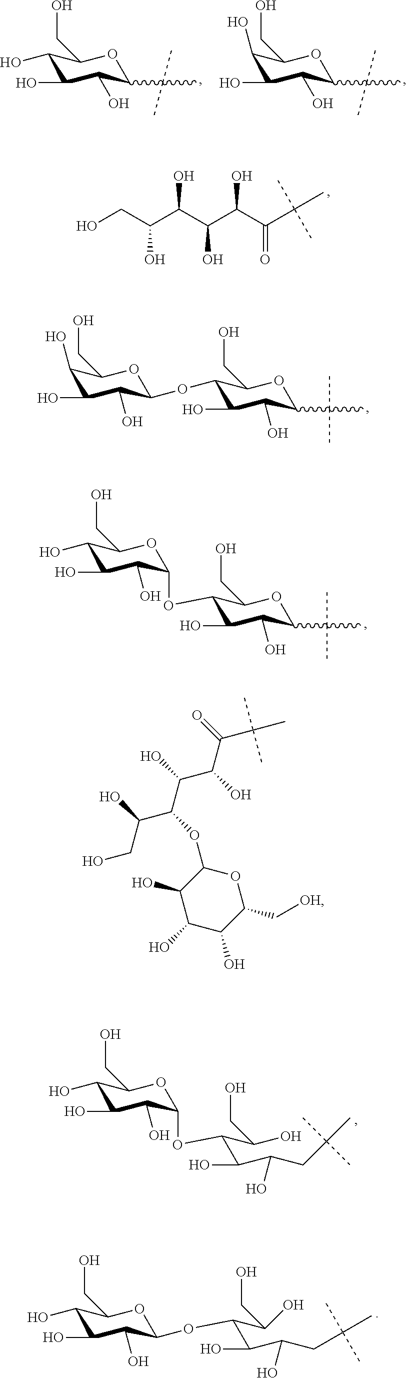

According to an embodiment, the present disclosure relates to compounds of formula I or its stereoisomers, prodrugs and pharmaceutically acceptable salts thereof:

wherein
R¹ is hydrogen;
R² is hydrogen;
L is a C₂-C₆ alkyl;

### X is O; and

Y is selected from the group consisting of

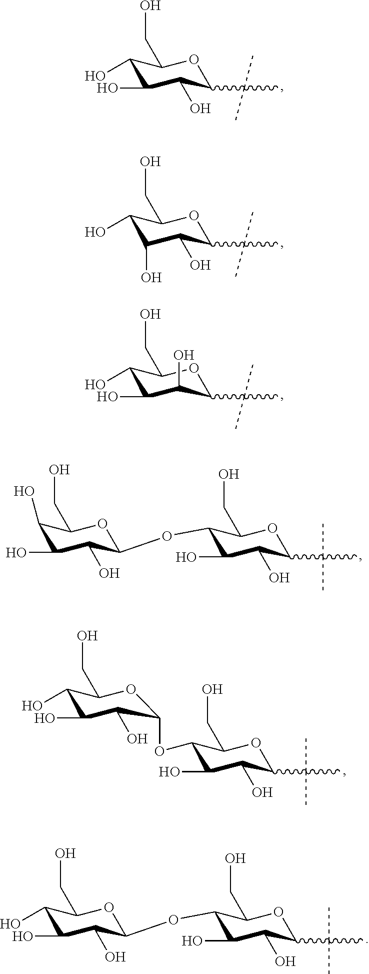

According to another embodiment, the present disclosure relates to compounds of formula I or its stereoisomers, prodrugs and pharmaceutically acceptable salts thereof:

wherein
R¹ is hydrogen;
R² is hydrogen;
L is a C₂-C₆ alkyl;

### X is NH; and

Y is selected from the group consisting of

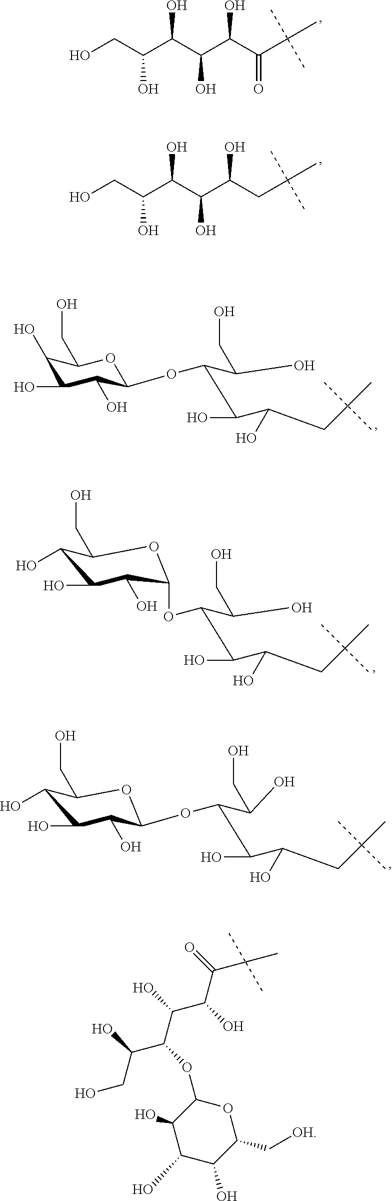

According to yet another embodiment, the present disclosure relates to compounds of formula I or its stereoisomers, prodrugs and pharmaceutically acceptable salts thereof:

wherein
R¹ is hydrogen;
R² is a C₂-C₁₂ alkyl; wherein alkyl is unsubstituted or substituted with two substituents independently selected from alkyl, halogen, hydroxy, monoalkylamino, dialkylamino, trialkylamino, nitro, and aryl;
L is a C₂-C₆ alkyl;

### X is NH; and

Y is selected from the group consisting of

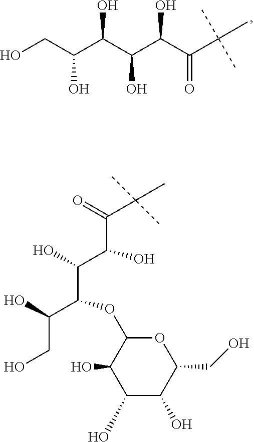

According to another embodiment, the present disclosure relates to compounds of formula I or its stereoisomers, prodrugs and pharmaceutically acceptable salts thereof:

wherein R¹ is hydrogen,
R² is selected from the group consisting of hydrogen, and a C₂-C₁₂ alkyl; wherein alkyl is unsubstituted or substituted with two substituents independently selected from alkyl, halogen, hydroxy, monoalkylamino, dialkylamino, trialkylamino, nitro, and aryl.
L is a C₂-C₆ alkyl;

### X is NH, and O;

Y is selected from the group consisting of

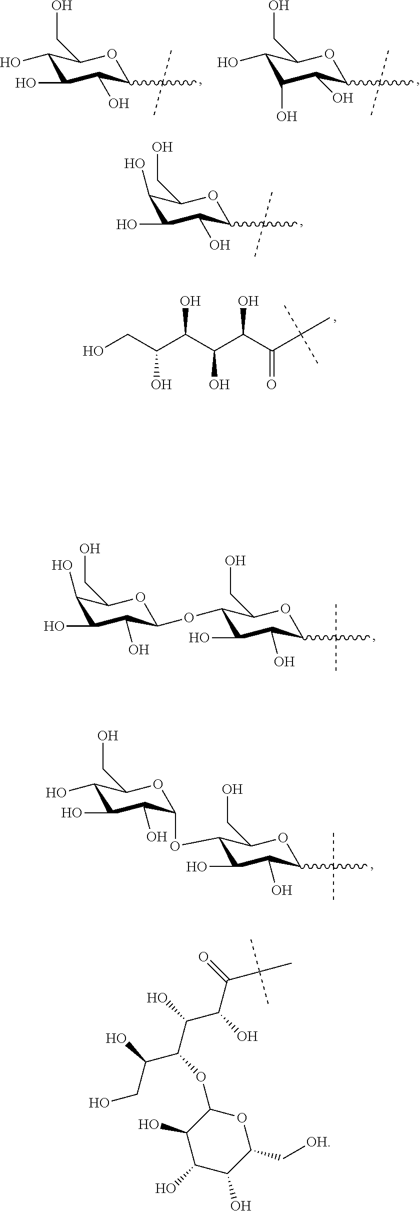

According to another embodiment, the present disclosure relates to compounds of formula I or its stereoisomers, prodrugs and pharmaceutically acceptable salts thereof:

wherein R¹ is hydrogen,
R² is selected from the group consisting of hydrogen, and a C₆-C₁₈ alkyl;
L is a C₂-C₆ alkyl;

### X is NH, and O;

Y is selected from the group consisting of

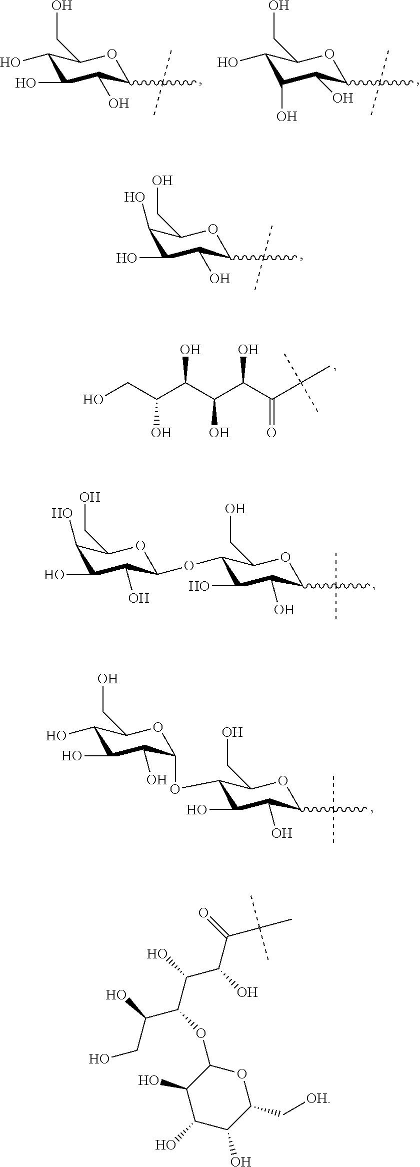

One embodiment of the present disclosure are compounds of formula I or its stereoisomers, prodrugs and pharmaceutically acceptable salts thereof, selected from the group consisting of,

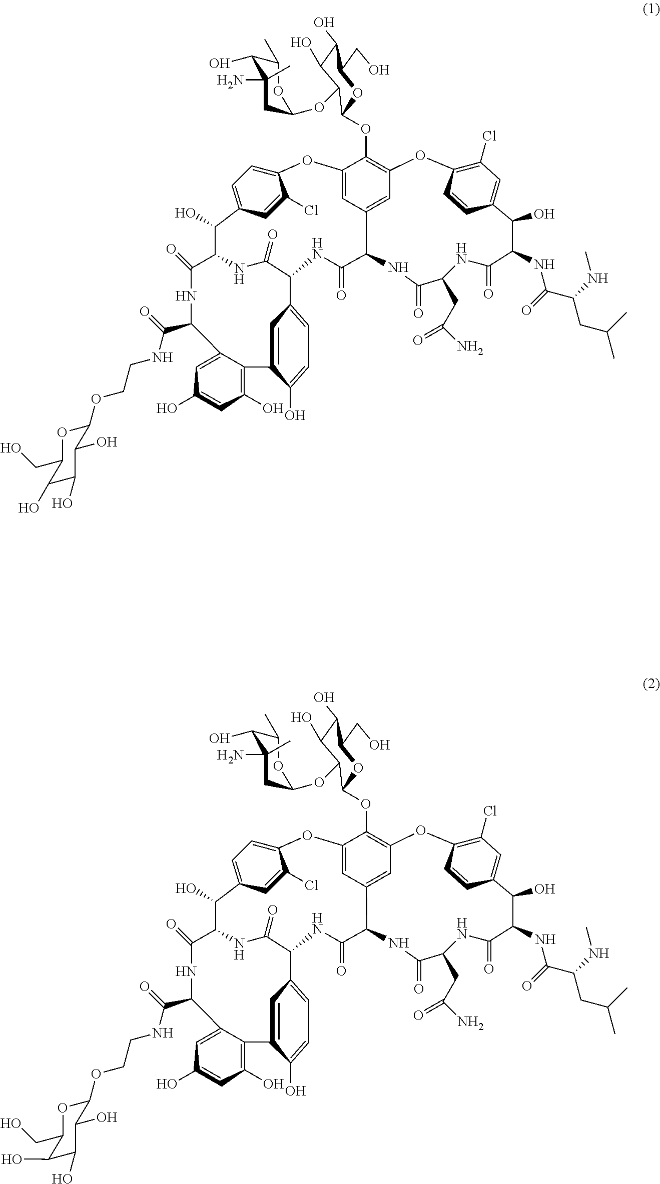

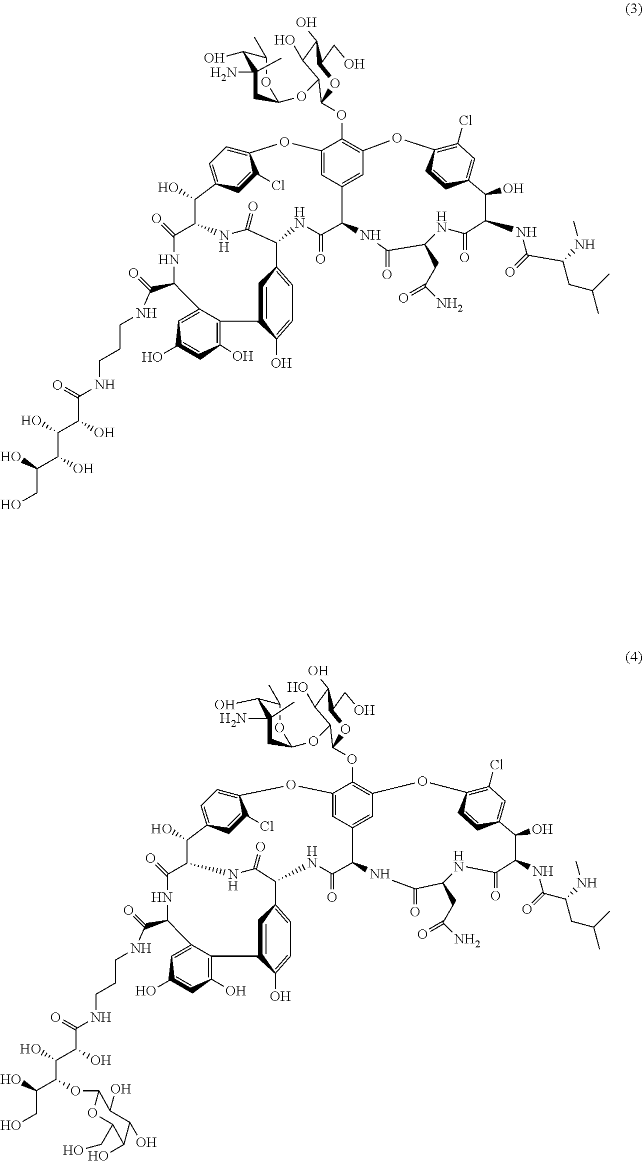

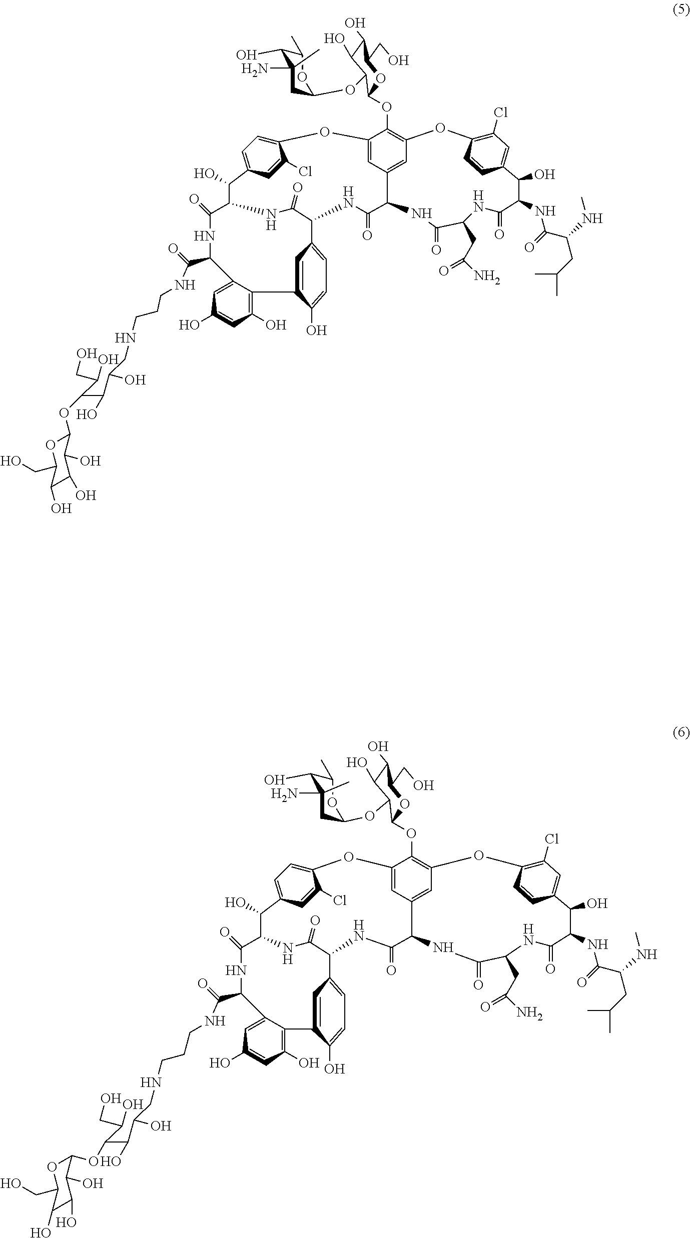

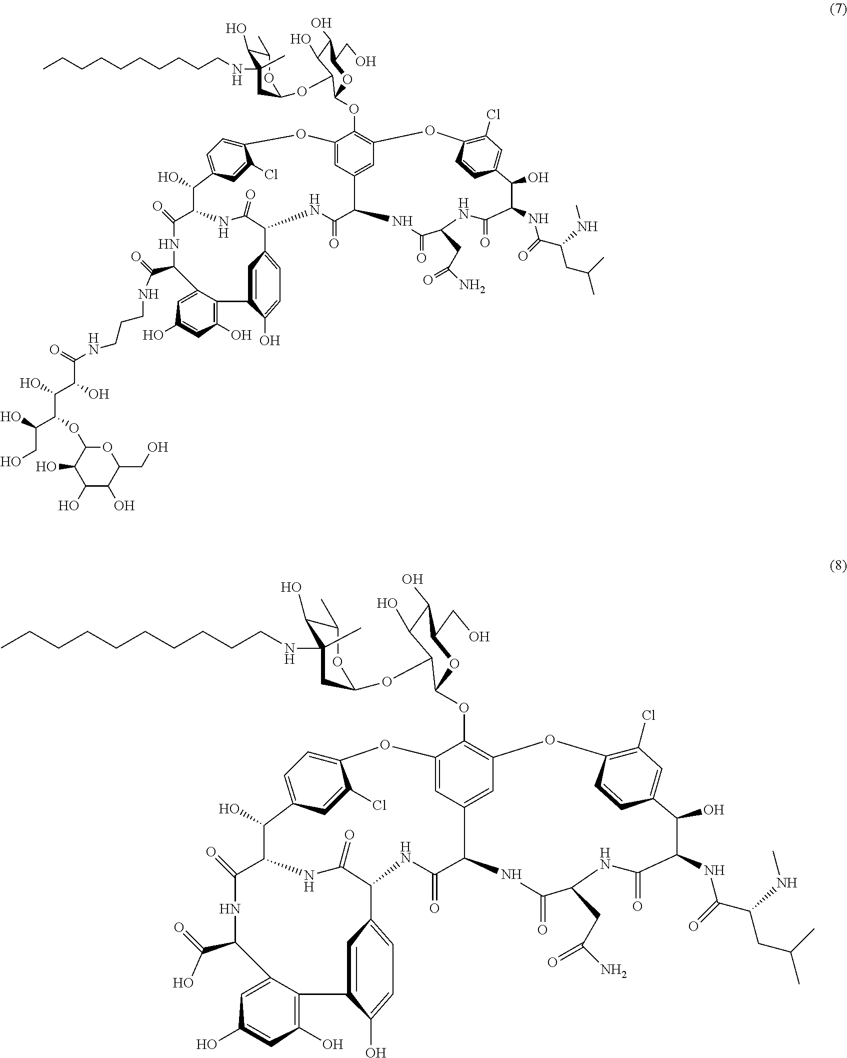

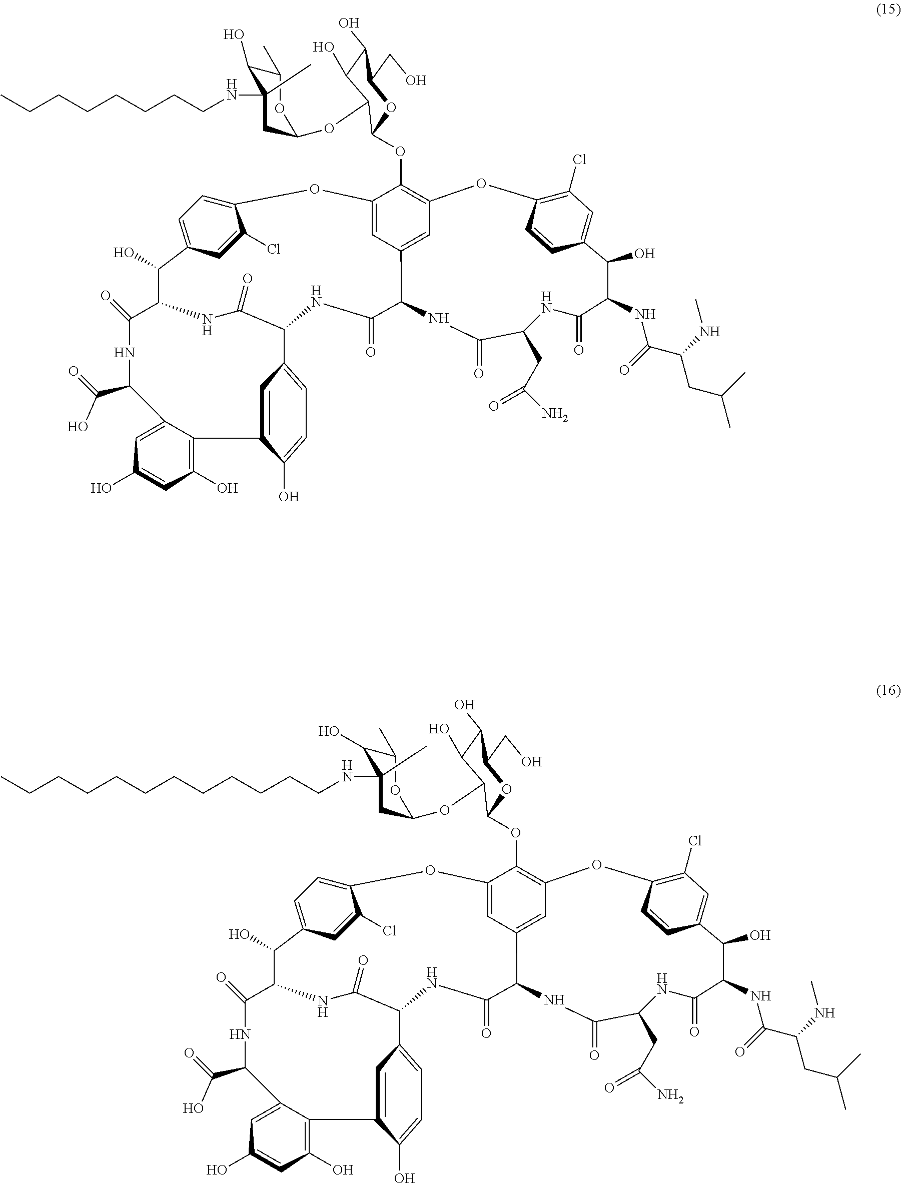

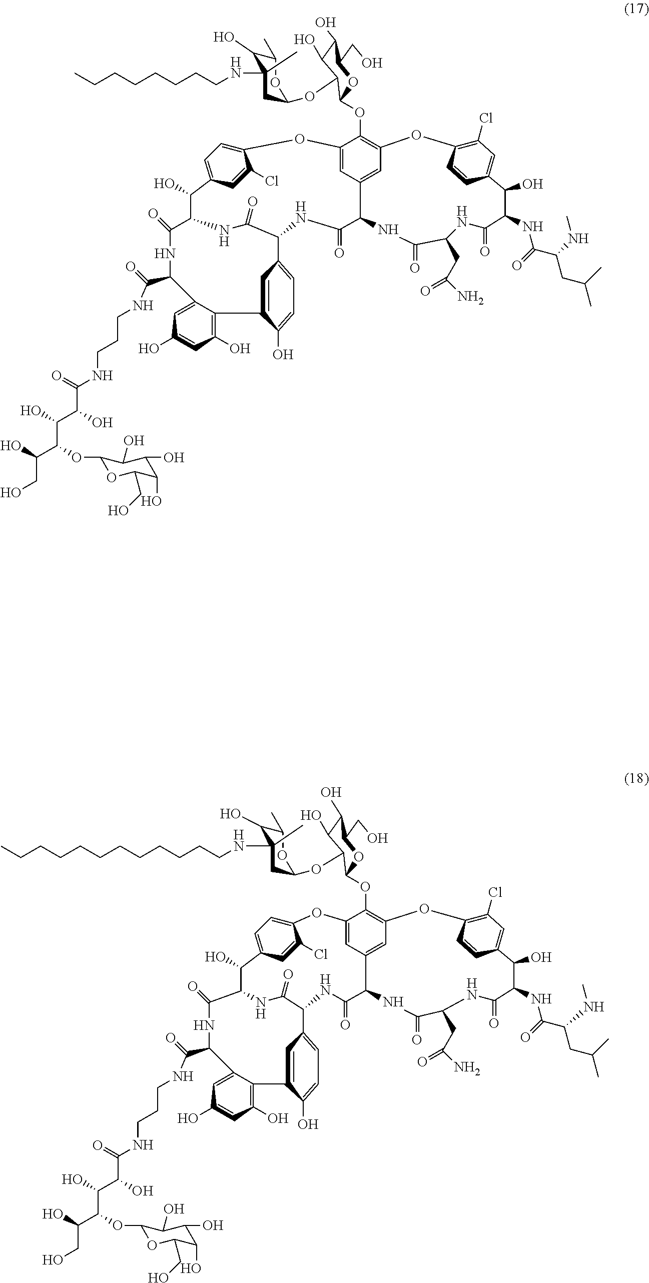

Particular embodiments of the present disclosure are compounds of formula I or its stereoisomers, prodrugs and pharmaceutically acceptable salts thereof, selected from the group consisting of,

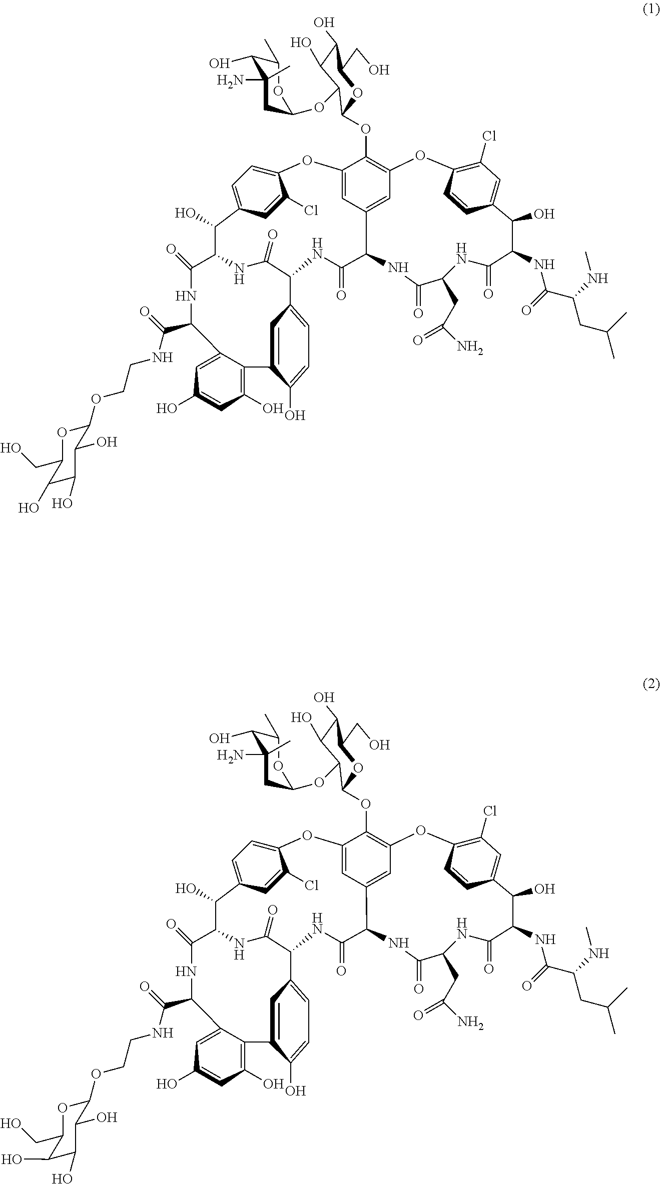

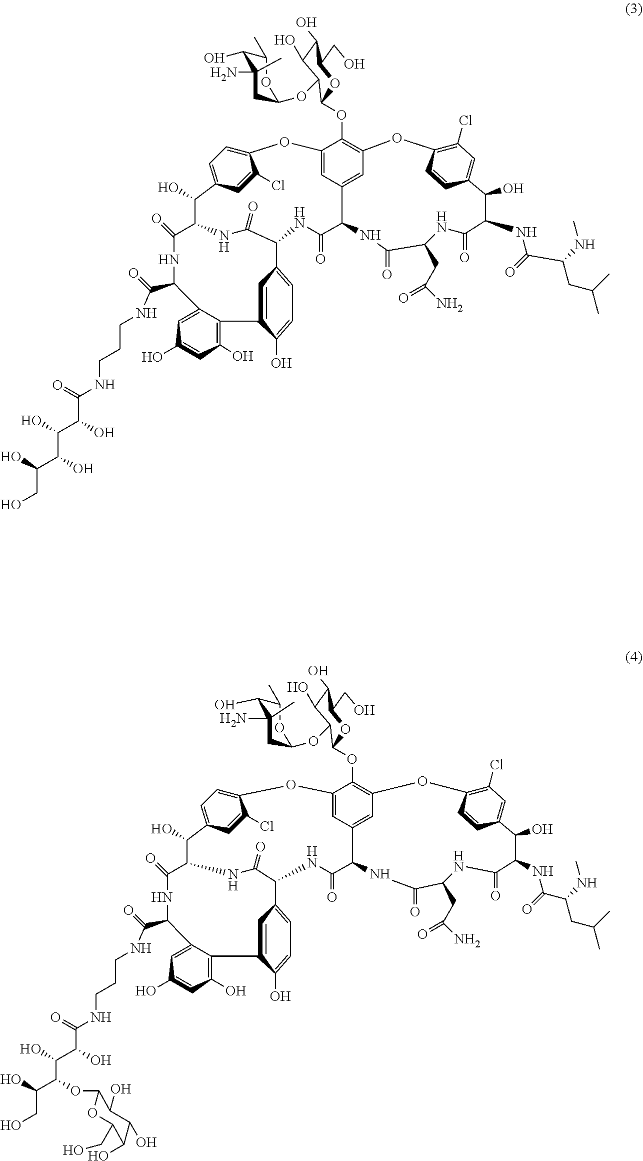

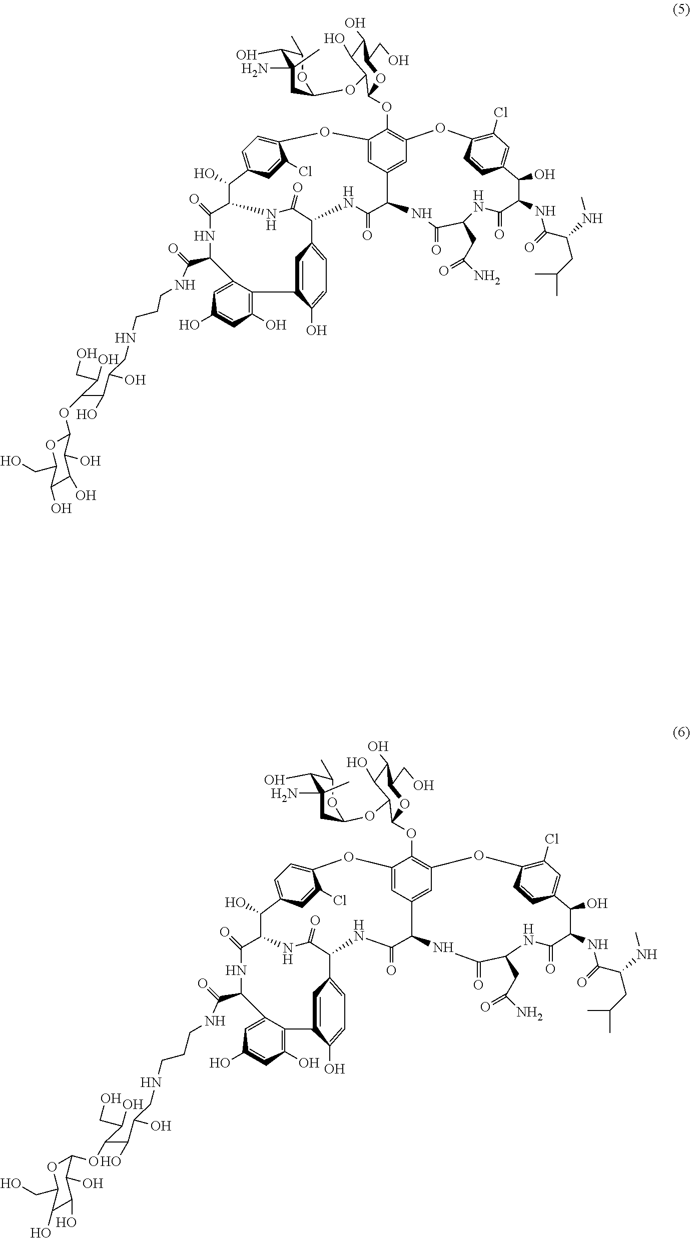

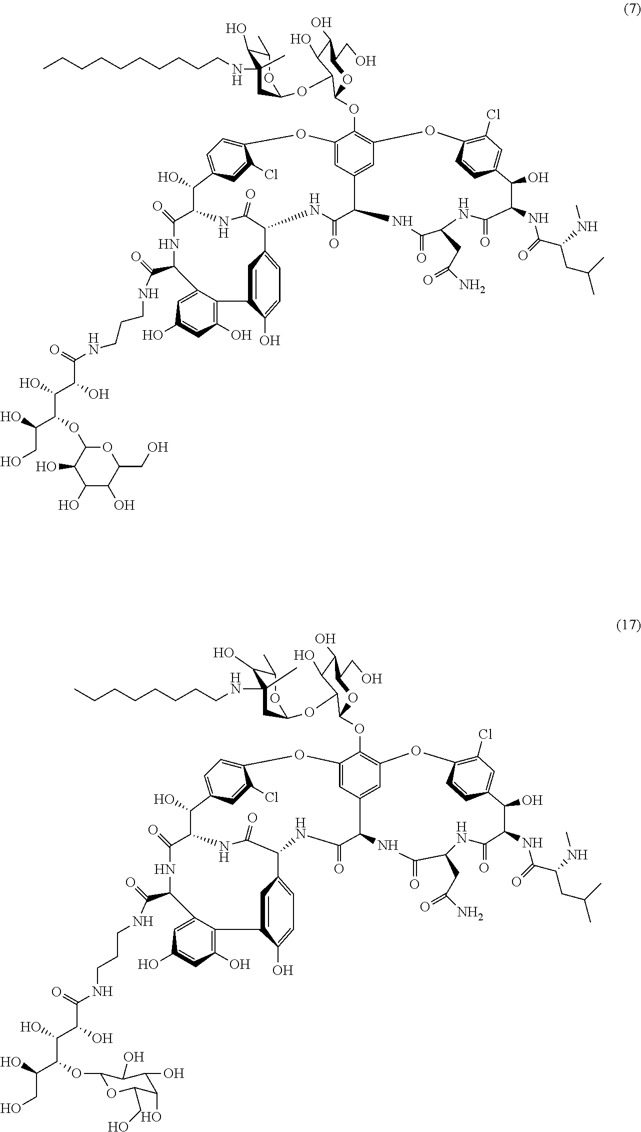

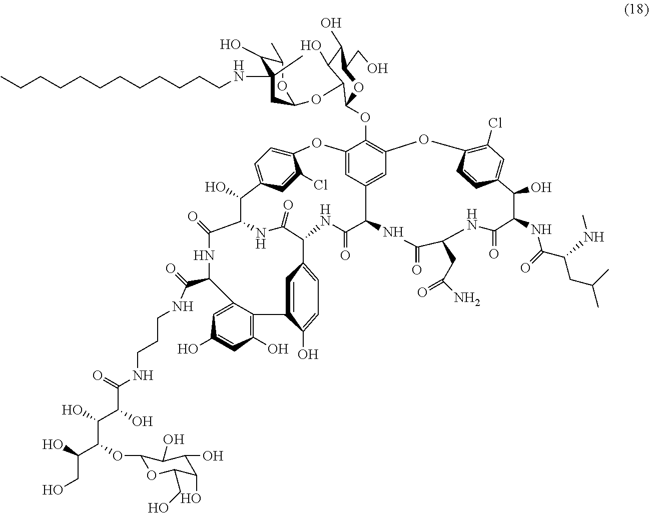

An embodiment of the present disclosure also relates to a compound of formula (I) or its stereoisomers, prodrugs and pharmaceutically acceptable salts thereof, for use as a medicament.

Another embodiment of the present disclosure also relates to a compound of formula (I) or its stereoisomers, prodrugs and pharmaceutically acceptable salts thereof, for use in treatment of a bacterial infection.

Yet another embodiment of the present disclosure also relates to a compound of formula (I) or its stereoisomers, prodrugs and pharmaceutically acceptable salts thereof, for use in the treatment of diseases caused by gram positive bacteria.

Another embodiment of the present disclosure relates to a pharmaceutical composition comprising a compound of formula (I) or pharmaceutically acceptable salts thereof, together with a pharmaceutically acceptable carrier and a method of preparing the same.

Yet another embodiment of the present disclosure relates to a pharmaceutical composition comprising a therapeutically effective amount of a compound of the present disclosure, alone or in combination with one or more pharmaceutically acceptable carriers.

An embodiment of the present disclosure relates to a method of killing a bacterial cell, the method comprising contacting the cell with a compound of formula (I) or its stereoisomers, prodrugs and pharmaceutically acceptable salts thereof, in an amount sufficient to kill the bacterial cell.

In an embodiment of the present disclosure the bacterial cell is selected from the group consisting of enterococci, staphylococci and streptococci.

The present disclosure describes vancomycin-sugar conjugates using facile synthetic methodology. These derivatives showed strong, broad-spectrum antibacterial activity and about >700 fold more active than parent drug, vancomycin against vancomycin-resistant E. faecium (VRE) and showed comparable or more active than vancomycin against methicillin-sensitive S. aureus (MSSA), methicillin-resistant S. aureus (MRSA), vancomycin-intermediate-resistant S. aureus (VISA), and vancomycin-sensitive E. faecium (VSE). These vancomycin-sugar conjugates are used to tackle bacterial infections.

An embodiment of the present disclosure also relates to a compound of formula (I) or its stereoisomers, prodrugs and pharmaceutically acceptable salts thereof, for use in treatment of a bacterial infection, wherein the bacterium comprises a vancomycin-resistant bacterium or a methicillin-resistant bacterium.

An embodiment of the present disclosure also relates to a compound of formula (I) or its stereoisomers, prodrugs and pharmaceutically acceptable salts thereof, for use in treatment of a bacterial infection, wherein the bacterium comprises a vancomycin-resistant Staphylococcus aureus, a vancomycin-resistant Enterococcus faecium or a methicillin-resistant Staphylococcus aureus.

Another embodiment of the disclosure includes a method of treatment of bacterial infection in a subject by administering to the subject an effective amount of the compound of formula I or its stereoisomers, prodrugs and pharmaceutically acceptable salts thereof.

The bacterial infection disclosed in the present disclosure is caused by a gram-positive bacterium.

The bacterial infection comprises an infection caused by a drug-resistant bacterium. The drug-resistant bacterium of the present disclosure is a vancomycin-resistant bacterium or a methicillin-resistant bacterium. The bacterium comprises a vancomycin-resistant Staphylococcus aureus, a vancomycin-resistant Enterococcus faecium or a methicillin-resistant Staphylococcus aureus.

A further embodiment of the present disclosure also relates to an article comprising: a composition comprising the compound of formula I or its stereoisomers, prodrugs and pharmaceutically acceptable salts thereof.

In an embodiment, the article comprises a substrate, wherein the substrate is coated with or impregnated with the composition comprising the compound of formula I or its stereoisomers, prodrugs and pharmaceutically acceptable salts thereof.

The compounds disclosed in the present disclosure showed antibacterial activity even up to 24 h in in-vivo time dependant whole blood assay, whereas vancomycin did not show any activity even at 3 h. Further, the compounds of the present disclosure have improved pharmacological properties as compared to parent compound, vancomycin.

The present disclosure further relates to a process of preparation of compounds of formula (I) or stereoisomers, prodrugs and pharmaceutically acceptable salts thereof.

The present subject matter further discloses a process for the preparation of vancomycin sugar conjugates of formula I. In an embodiment, the sugar conjugates of vancomycin of the present subject matter were synthesized by coupling carboxylic group of vancomycin with cyclic and acyclic sugar moieties through amide coupling using at least one organic solvent and coupling agent. Further, the reaction is carried out between 0° C.—room temperature. In one embodiment, the coupling agent is o-benzotriazole-N,N,N′,N′-tetramethyl-uronium-hexafluorophosphate (HBTU). Other coupling agents such as 2-(1H-7-azabenzotriazol-1-yl)-1,1,3,3-tetramethyl uronium hexafluorophosphate Methanaminium (HATU), N,N′-diisopropylcarbodiimide (DIC), 1-ethyl-3-(3-dimethylaminopropyl carbodiimide (EDCI) and O-(benzotriazol-1-yl)-N,N,N′,N′-tetramethyluronium tetrafluoroborate (TBTU) can be used instead of HBTU. The reaction mixture should be cooled to 0° C., and 1.5 equivalents of amide coupling reagent (HBTU) in DMF should be added followed by (5.0 equivalents) of diisopropylethylamine (DIPEA) and then appropriate amine should be added for amide coupling. The reaction mixture was then allowed to warm to room temperature (25° C.) and stirred for 8-12 h. In another embodiment, the organic solvent includes at least one selected from the group of dimethylformamide (DMF), dimethyl sulfoxide, and others as understood by a person skilled in the art.

In an embodiment, the synthesized compounds are further characterized by IR, ¹H-NMR, ¹³C-NMR and HR-MS.

### Abbreviations

The following abbreviations are employed in the examples and elsewhere herein:

### DCM: Dichloromethane,

NaN₃: Sodium azide,

### CH₃OH: Methanol,

NaOMe: Sodium methoxide,
PPh₃: Triphenyl phosphine,

### DMF: N,N-Dimethylformamide,

DMSO: Dimethyl sulfoxide,
HBTU: Benzotriazole-N,N,N′,N′-tetramethyl-uronium-hexafluorophosphate,

### DIPEA: Diisopropylethylamine,

HCl: Hydrochloric acid,

### IPA: Isopropanol,

NaBH₄: Sodium borohydride,
NaCNBH₃: Sodium cyanoborohydride
RT: Room temperature,

### μM: Micromolar.

### EXAMPLES

The disclosure is further illustrated by the following examples which in no way should be construed as being further limiting. One skilled in the art will readily appreciate that the specific methods and results described are merely illustrative.

### Example 1

### Preparation of (1)

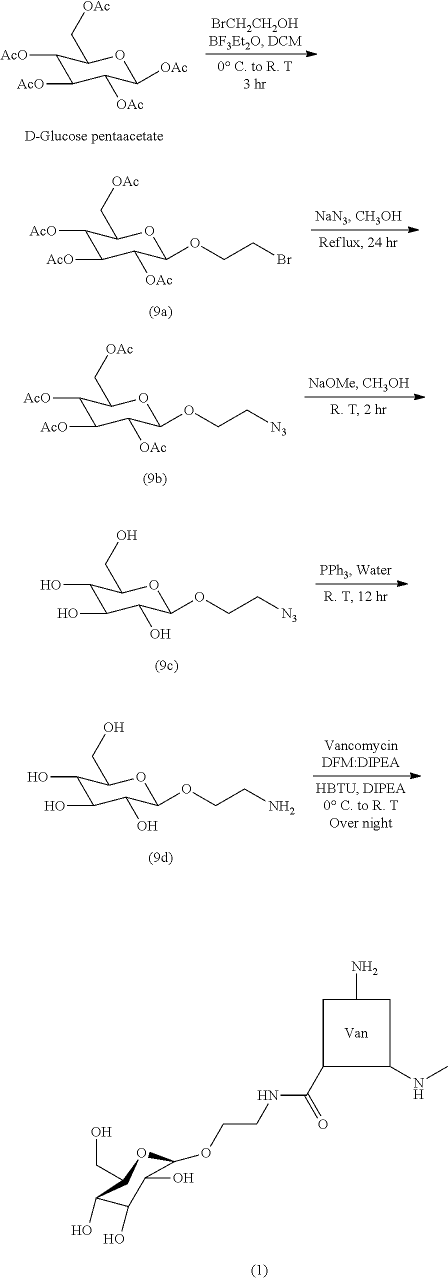

### Synthesis of 9a

About 1.0 g of D-glucose pentaacetate was dissolved in about 10 mL of dry DCM. Then about 1.3 mL (1.2 equivalents) of BF₃.Et₂O was added to the reaction mixture drop wise followed by another 0.22 mL (1.2 equivalents) of 2-bromoethanol. The reaction mixture was stirred at 0° C. for 3 h, and then stirred at room temperature for overnight. About 0.53 g (1.5 equivalents) of potassium carbonate was added 30 min before the reaction was stopped. Then the crude solution was extracted with chloroform and purified through silica gel column chromatography (EtOAc/Hexane 30:70) to get pure 9a with 79% yield. ¹H-NMR (400 MHz, CDCl₃) δ/ppm: 4.573 (d, 1H), 4.236-4.123 (m, 6H), 3.704 (m, 2H), 3.458 (m, 2H), 2.026 (s, 12H). 13C-NMR (100 MHz, CDCl3) δ/ppm: 170.04, 100.12, 71.88, 71.21, 70.05, 67.25, 67.43, 60.56, 29.76, 19.82. HRMS: m/z 477.0351 (observed); 477.0372 (calculated for M+Na⁺).

### Synthesis of 9b:

About 0.52 g of 9a was dissolved in about 10 mL of methanol, and then about 0.37 g (2.0 equivalents) of sodium azide was added to the reaction mixture. Now, the reaction mixture was refluxed at 70° C. for 24 h. Then the crude solution was extracted with chloroform and purified through silica gel column chromatography (EtOAc/Hexane 30:70) to get pure 9b with 86% yield. FT-IR (NaCl): 2950 cm⁻¹ (—CH₂— asym. str.), 2884 cm⁻¹ (—CH₂ sym. str.), 2106 cm⁻¹ (—N₃ str.), 1754 cm⁻¹ (—OAc C═O str.). ¹H-NMR (400 MHz, CDCl₃) δ/ppm: 4.564 (d, 1H), 4.238-4.109 (m, 6H), 3.490 (m, 2H), 3.292 (m, 2H), 2.018 (s, 12H). 13C-NMR (100 MHz, CDCl₃) δ/ppm: 169.36, 99.78, 71.90, 71.06, 70.18, 67.64, 67.45, 60.95, 49.63, 19.77. HRMS: m/z 440.1278 (observed); 440.1281 (calculated for M+Na⁺).

### Synthesis of 9c:

About 0.3 g of 9b was dissolved in 5 mL of methanol, and then about 0.165 g (4.0 equivalents) of sodium methoxide was added to the reaction mixture and reaction was stirred for 2 h at room temperature. Then to the reaction mixture, dowex resin (strongly acidic) was added and pH of the reaction mixture was adjusted to 6. Now the reaction mixture was filtered and the filtrate was evaporated to get 9c with quantitative yield. FT-IR (NaCl): 3364 cm⁻¹ (—OH str.), 2929 cm⁻¹ (—CH₂— asym. str.), 2885 cm⁻¹ (—CH₂— sym. str.), 2105 cm-1 (—N₃ str.). ¹H NMR (400 MHz, DMSO-d6) δ/ppm: 4.184 (d, 1H), 3.882-3.416 (m, 6H), 3.112 (m, 2H), 2.990 (m, 2H). ¹³C-NMR (100 MHz, DMSO-d6) δ/ppm: 103.00, 76.99, 76.77, 73.43, 70.11, 67.37, 61.14, 50.43. HRMS: m/z 272.0844 (observed); 272.0859 (calculated for M+Na⁺).

### Synthesis of 9d:

About 0.15 g of 9c was dissolved in about 1:1 methanol/water. Then about 0.24 g (1.5 equivalents) of triphenyl phosphine was added to the reaction mixture and the reaction mixture was refluxed at 70° C. for 12 h. Now the crude solution was extracted with water and it was kept in the lyophilizer to get pure and dry 9d with 75% yield. FT-IR (NaCl): 3322 cm⁻¹ (—OH and —NH2 asym, sym. str.), 2929 cm⁻¹ (—CH₂— asym. str.), 2890 cm⁻¹ (—CH₂— sym. str.). ¹H-NMR (400 MHz, DMSO-d6) δ/ppm: 4.559 (d, 1H), 4.172-3.771 (m, 6H), 3.276 (m, 2H), 3.183 (t, 2H). ¹³C-NMR (100 MHz, DMSO-d6) δ/ppm: 104.52, 78.32, 77.96, 75.40, 71.92, 68.19, 59.95, 43.62. HRMS: m/z 224.1122 (observed); 224.1134 (calculated for M+H⁺)

### Synthesis of 1:

Vancomycin hydrochloride (100 mg, 67 μmol) was dissolved in 1:1 mixture of dry dimethyl formamide (1 mL). To this two equivalents of 9d in 1 mL of dry dimethylformamide was added. The reaction mixture was cooled to about 0° C., and about 0.22 mL (1.5 equivalents) of 0.45 M benzotriazole-N,N,N′,N′-tetramethyl-uronium-hexafluorophosphate (HBTU) solution in DMF was added followed by about 58 μL (5.0 equivalents) of diisopropylethylamine (DIPEA). The reaction mixture was then allowed to warm to room temperature and stirred for about 8-12 h. The product was purified, by preparative reversed-phase HPLC using about 0.1% trifluoro acetic acid in H₂O/acetonitrile mixture and then lyophilized to afford tris-(trifluoroacetate) salts of final compounds (50-55 μmol, 75-80%). These vancomycin-sugar conjugates were purified and characterized by ¹H-NMR and HR-MS (Table 1). The purification was done by preparative reverse phase HPLC using 0.1% Trifluoro acetic acid (TFA) in water/acetonitrile (0-100%) as mobile phase. C18 column (10 mm diameter, 250 mm length) and UV detector (at 270 nm wave length) were used. The collected fractions, from HPLC were frozen by liquid N₂ and lyophilized in freeze dryer.

### Example 2

### Preparation of (2)

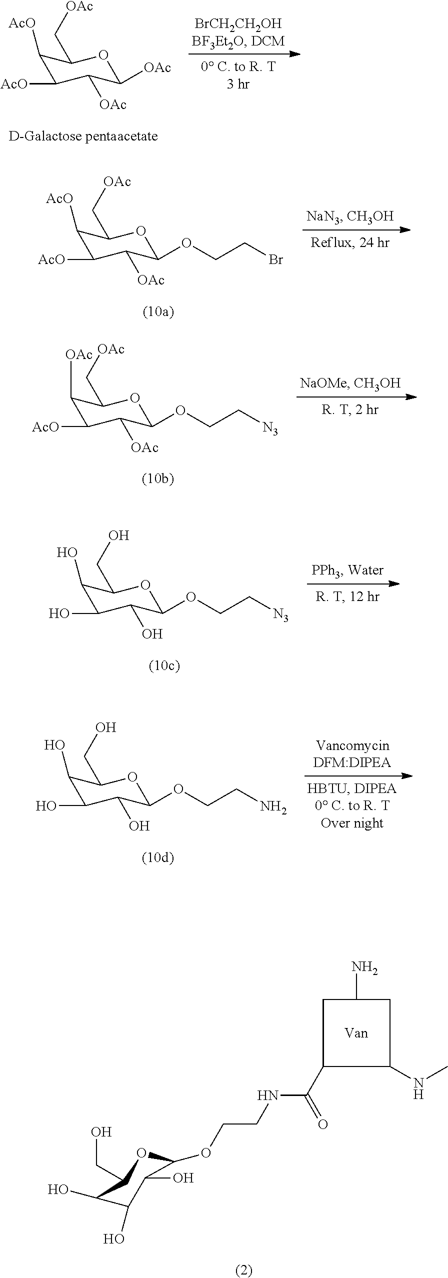

### Synthesis of 10a:

About 2.5 g of D-galactose pentaacetate was dissolved in about 20 mL of dry DCM. Then about 3.63 mL (1.2 equivalents) of BF₃.Et₂O was added to the reaction mixture drop wise followed by another about 0.54 mL (1.2 equivalents) of 2-bromoethanol. The reaction mixture was stirred at 0° C. for 3 h, stirred at room temperature for overnight. About 1.33 g (1.5 equivalents) of potassium carbonate was added 30 min before the reaction was stopped. Then the crude solution was extracted with chloroform and purified through silica gel column chromatography (EtOAc/Hexane 30:70) to get pure 10a with 70% yield. ¹H-NMR (400 MHz, CDCl₃) δ/ppm: 4.523 (d, 1H), 4.314-3.809, (m, 6H), 3.471 (m, 4H), 2.060 (s, 12H). ¹³C-NMR (100 MHz, CDCl₃) δ/ppm: 169.75, 100.43, 72.05, 71.23, 69.56, 68.70, 67.22, 61.05, 29.99, 22.12. HRMS: m/z 477.0351 (observed); 477.0372 (calculated for M+Na⁺).

### Synthesis of 10b:

About 1.0 g of 10a was dissolved in 20 mL of methanol, then about 0.729 g (2 equivalents) of sodium azide was added to the reaction mixture. Now, the reaction mixture was refluxed at 70° C. for 24 h. Then the crude solution was extracted with chloroform and purified through silica gel column chromatography (EtOAc/Hexane 30:70) to get pure 10b with 60% yield. FT-IR (NaCl): 2940 cm⁻¹ (—CH₂— asym. str.), 2885 cm-1 (—CH₂— sym. str.), 2102 cm⁻¹ (—N₃ str.), 1742 cm⁻¹ (—OAc C═O str.). ¹H-NMR (400 MHz, CDCl₃) δ/ppm: 4.554 (d, 1H), 4.238-3.905 (m, 6H), 3.490 (m, 2H), 3.292 (m, 2H), 2.018 (s, 12H). ¹³C-NMR (100 MHz, CDCl₃) δ/ppm: 170.37, 101.30, 71.06, 70.99, 68.70, 68.17, 67.17, 61.41, 50.72, 20.80. HRMS: m/z 440.1274 (observed) 440.1281 (calculated for M+Na⁺).

### Synthesis of 10c:

About 0.085 g of 10b was dissolved in 3 mL of methanol, then about 0.04 g (4.0 equivalents) of sodium methoxide was added to the reaction mixture and reaction mixture was stirred for 2 h with stirring at room temperature. Then to the reaction mixture, dowex resin (strongly acidic) was added and pH of the reaction mixture was adjusted at about 6. Now the reaction mixture was filtered and the filtrate was evaporated to get 10c with 98% yield. FT-IR (NaCl): 3394 cm⁻¹ (—OH str.), 2923 cm⁻¹ (—CH₂— asym. str.), 2885 cm⁻¹ (—CH₂— sym. str.), 2105 cm⁻¹ (—N₃ str.). ¹H-NMR (400 MHz, DMSO-d6) δ/ppm: 4.127 (d, 1H), 3.845-3.456 (m, 6H), 3.296 (m, 4H). ¹³C-NMR (100 MHz, DMSO-d6) δ/ppm: 103.62, 75.37, 73.55, 70.52, 68.02, 67.15, 60.38, 50.50. HRMS: m/z 272.0844 (observed); 272.0859 (calculated for M+Na⁺).

### Synthesis of 10d:

About 50 mg of 10c was dissolved in 1:1 methanol/water. Then about 79 mg (1.5 equivalents) of triphenylphosphine was added to the reaction mixture and the reaction mixture was refluxed at 70° C. for 12 h. Now the crude solution was extracted with water and it was kept in the lyophilizer to get pure and dry 10d with 75% yield. FT-IR (NaCl) 3329 cm⁻¹ (—OH and —NH₂ asym., sym. str.), 2927 cm⁻¹ (—CH₂— asym. str.) 2885 cm⁻¹ (—CH₂— sym. str.). ¹H-NMR (400 MHz, DMSO-d6) δ/ppm: 4.449 (d, 1H), 4.047-3.566 (m, 6H), 3.699 (m, 2H), 3.058 (t, 2H). ¹³C-NMR (100 MHz, DMSO-d6) δ/ppm: 103.85, 76.27, 74.22, 71.12, 69.09, 67.98, 61.34, 51.19. HRMS: m/z 224.1119 (observed); 224.1134 (calculated for M+Na⁺).

### Synthesis of 2:

Vancomycin hydrochloride (100 mg, 67 μmol) was dissolved in 1:1 mixture of dry dimethyl formamide (1 mL) and dry dimethyl sulfoxide (1 mL). To this two equivalents of 10d in 1 mL of dry dimethylformamide was added. The reaction mixture was cooled to about 0° C., and about 0.22 mL (1.5 equivalents) of 0.45 M benzotriazole-N,N,N′,N′-tetramethyl-uronium-hexafluorophosphate (HBTU) solution in DMF was added followed by about 58 μL (5.0 equivalents) of diisopropylethylamine (DIPEA). The reaction mixture was then allowed to warm to room temperature and stirred for about 8-12 h. The product was purified by preparative reversed-phase HPLC using about 0.1% trifluoro acetic acid in H₂O/acetonitrile mixture and then lyophilized to afford tris-(trifluoroacetate) salts of final compounds (50-55 μmol, 75-80%). These vancomycin-sugar conjugates were purified and characterized by ¹H-NMR and HR-MS (Table 1). The purification was done by preparative reverse phase HPLC using 0.1% trifluoro acetic acid (TFA) in water/acetonitrile (0-100%) as mobile phase. C18 column (10 mm diameter, 250 mm length) and UV detector (at 270 nm wave length) were used. The collected fractions, from HPLC were frozen by liquid N₂ and lyophilized in freeze dryer.

### Example 3

### Preparation of (3)

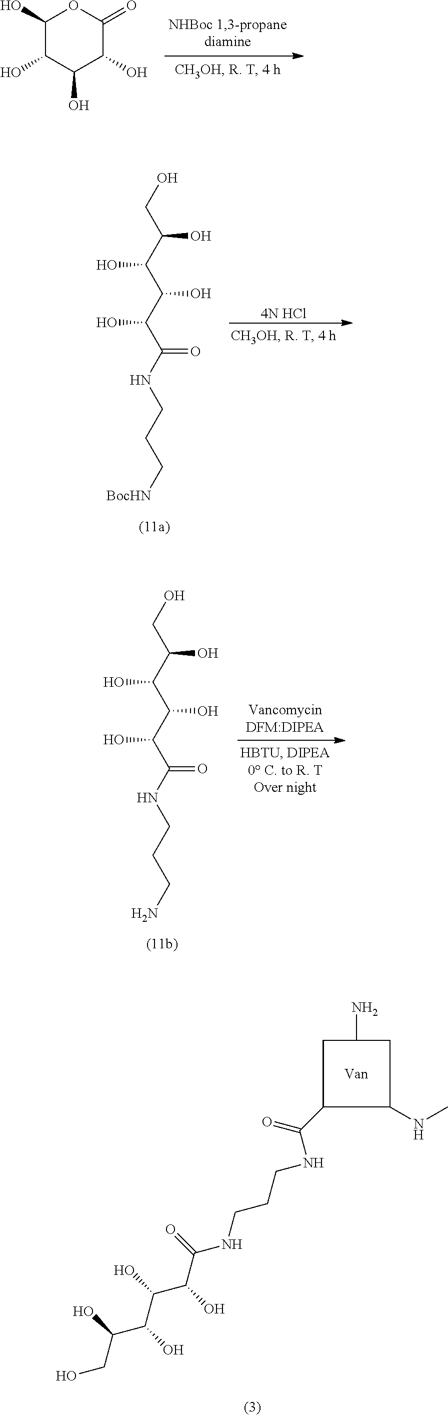

### Synthesis of 11a:

About 2.0 g of D-gluconicacid lactone was dissolved in 12 mL of methanol, then about 2.3 g (1.2 equivalents) of N-Boc-1,3-propanediamine was added to the reaction mixture. Now the reaction mixture was refluxed at 70° C. for 24 h. Then methanol was removed by rotary evaporator, the residue was washed with ethyl acetate and finally with chloroform. Then it was kept in high vacuum oven for overnight to get the pure and dry 11a with 98% yield. FT-IR (NaCl): 3329 cm⁻¹ (—OH str.), 2933 cm⁻¹ (—CH₂— asym. str.), 2882 cm⁻¹ (—CH₂— sym. str.), 1687 cm⁻¹ (Amide-I C═O str.), 1654 cm⁻¹ (Amide-II —NH— ben.). ¹H-NMR (400 MHz, DMSO-d6) δ/ppm: 4.483-3.473 (m, 4H), 4.358-3.572 (m, 2H), 2.927-3.077 (m, 4H), 1.495 (m, 2H), 1.374 (s, 9H). ¹³C-NMR (100 MHz, DMSO-d6) δ/ppm: 173.16, 156.24, 78.18, 73.92, 72.72, 71.83, 70.84, 63.62, 37.54, 36.15, 29.83, 28.59. HRMS: m/z 375, 1726 (observed); 375.1743 (calculated for M+Na⁺).

### Synthesis of 11b:

About 2.56 g of 11a was dissolved in 5 mL of methanol and 5 mL of 4N HCl was added to it. Then it was stirred for 4 h at room temperature. Then solvent was evaporated to get pure and dry 11b with 96% yield. FT-IR (NaCl): 3335 cm⁻¹ (—OH and —NH₂ sym., asym. str.), 2927 cm⁻¹ (—CH₂— asym. str.), 2886 cm⁻¹ (—CH₂— sym. str.). ¹H-NMR (400 MHz, DMSO-d6) δ/ppm: 4.230-3.531 (m, 4H), 4.124-3.794 (m, 2H), 2.881 (t, 4H), 1.905 (m, 2H). ¹³CNMR (400 MHz, DMSO-d6) δ/ppm: 174.34, 80.41, 74.03, 72.65, 69.15, 62.92, 60.31, 36.20, 25.13. HRMS: m/z 253.1381 (observed); 253.1400 (calculated for M+H⁺).

### Synthesis of 3:

Vancomycin hydrochloride (100 mg, 67 μmol) was dissolved in 1:1 mixture of dry dimethyl formamide (1 mL) and dry dimethyl sulfoxide (1 mL). To this two equivalents of 11b in 1 mL of dry dimethylformamide was added. The reaction mixture was cooled to about 0° C., and about 0.22 mL (1.5 equivalents) of 0.45 M benzotriazole-N,N,N′,N′-tetramethyl-uronium-hexafluorophosphate (HBTU) solution in DMF was added followed by about 58 μL (5.0 equivalents) of diisopropylethylamine (DIPEA). The reaction mixture was then allowed to warm to room temperature and stirred for about 8-12 h. The product was purified by preparative reversed-phase HPLC using about 0.1% trifluoro acetic acid in H₂O/acetonitrile mixture and then lyophilized to afford tris-(trifluoroacetate) salts of final compounds (50-55 μmol, 75-80%). These vancomycin-sugar conjugates were purified and characterized by ¹H-NMR and HR-MS (Table 1). The purification was done by preparative reverse phase HPLC using 0.1% trifluoro acetic acid (TFA) in water/acetonitrile (0-100%) as mobile phase. C18 column (10 mm diameter, 250 mm length) and UV detector (at 270 nm wave length) were used. The collected fractions, from HPLC were frozen by liquid N₂ and lyophilized in freeze dryer.

### Example 4

### Preparation of 4

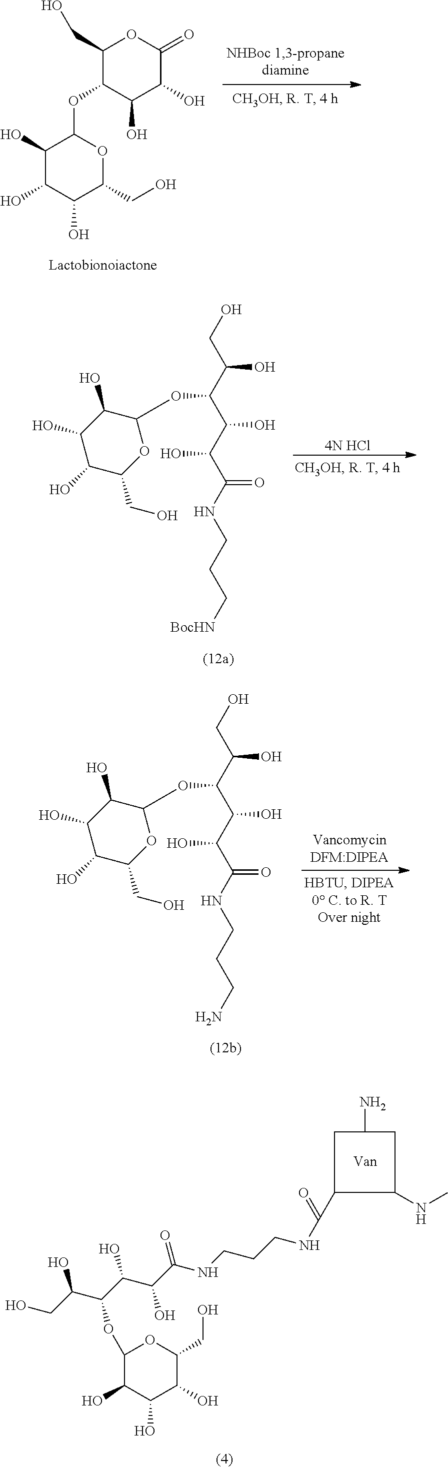

### Synthesis of 12a:

About 1.3 g of lactonobionolactone was dissolved in 5 mL of methanol, then about 0.89 g (1.2 equivalents) of N-Boc-1,3-propanediamine was added to the reaction mixture. Now the reaction mixture was refluxed at 70° C. for 24 h. Then methanol was removed by rotavapour, the residue was washed with ethyl acetate and finally with chloroform. Then it was kept in high vacuum oven for overnight to get the pure and dry 12a with 72% yield. FT-IR (NaCl): 3341 cm⁻¹ (—OH str.), 2929 cm⁻¹ (—CH₂— asym. str.), 2888 cm⁻¹ (—CH₂— sym. str.), 1685 cm⁻¹ (Amide-I C═O str.), 1660 cm⁻¹ (Amide-II —NH— ben.). ¹H-NMR (400 MHz, DMSO-d6) δ/ppm: 4.576 (d, 1H), 4.200-3.579 (m 12H), 3.300 (t, 2H), 3.118 (t, 2H), 1.719 (Q, 2H), 1.446 (s, 9H). ¹³C-NMR (100 MHz. DMSO-d6) δ/ppm: 171.96, 170.34, 103.15, 81.23, 73.23, 71.44, 69.13, 68.56, 62.27, 49.76, 36.21, 25.98, 21.02. HRMS: m/z 515.2489 (observed); 515.2452 (calculated for M+H⁺).

### Synthesis of 12b:

About 1.35 g of 12a was dissolved in 5 mL of methanol and 5 mL of 4N HCl was added to it. Then it was stirred for 4 h at room temperature. Then solvent was evaporated to get pure and dry compound 12b with 89% yield. FT-IR (NaCl): 3297 cm⁻¹ (—OH and —NH₂ sym., asym. str.), 2932 cm⁻¹ (—CH₂— asym. str.), 2888 cm⁻¹ (—CH₂— sym. str.), 1685 cm⁻¹ (Amide-I C═O str.), 1648 cm⁻¹ (Amide-II —NH— ben.). ¹H-NMR (400 MHz, DMSO-d6) δ/ppm: 4.572 (d, 1H), 4.411-3.576 (m, 12H), 3.352 (t, 2H), 3.303 (t, 2H), 1.721 (Q, 2H). ¹³C-NMR (100 MHz, DMSO-d6) δ/ppm: 172.74, 103.12, 81.35, 73.30, 71.58, 69.10, 68.01, 62.84, 49.60, 36.05, 25.05. HRMS: m/z 415.1901 (observed); 415.1928 (calculated for M+H⁺).

### Synthesis of 4:

Vancomycin hydrochloride (100 mg, 67 μmol) was dissolved in 1:1 mixture of dry dimethyl formamide (1 mL) and dry dimethyl sulfoxide (1 mL). To this two equivalents of 12b in 1 mL of dry dimethylformamide was added. The reaction mixture was cooled to about 0° C., and about 0.22 mL (1.5 equivalents) of 0.45 M benzotriazole-N,N,N′,N′-tetramethyl-uronium-hexafluorophosphate (HBTU) solution in DMF was added followed by about 58 μL (5.0 equivalents) of diisopropylethylamine (DIPEA). The reaction mixture was then allowed to warm to room temperature and stirred for about 8-12 h. The product was purified by preparative reversed-phase HPLC using about 0.1% trifluoro acetic acid in H₂O/acetonitrile mixture and then lyophilized to afford tris-(trifluoroacetate) salts of final compounds (50-55 μmol, 75-80%). These vancomycin-sugar conjugates were purified and characterized by ¹H-NMR and HR-MS (Table 1). The purification was done by preparative reverse phase HPLC using 0.1% trifluoro acetic acid (TFA) in water/acetonitrile (0-100%) as mobile phase. C18 column (10 mm diameter, 250 mm length) and UV detector (at 270 nm wave length) were used. The collected fractions, from HPLC were frozen by liquid N₂ and lyophilized in freeze dryer.

### Example 5

### Preparation of 5

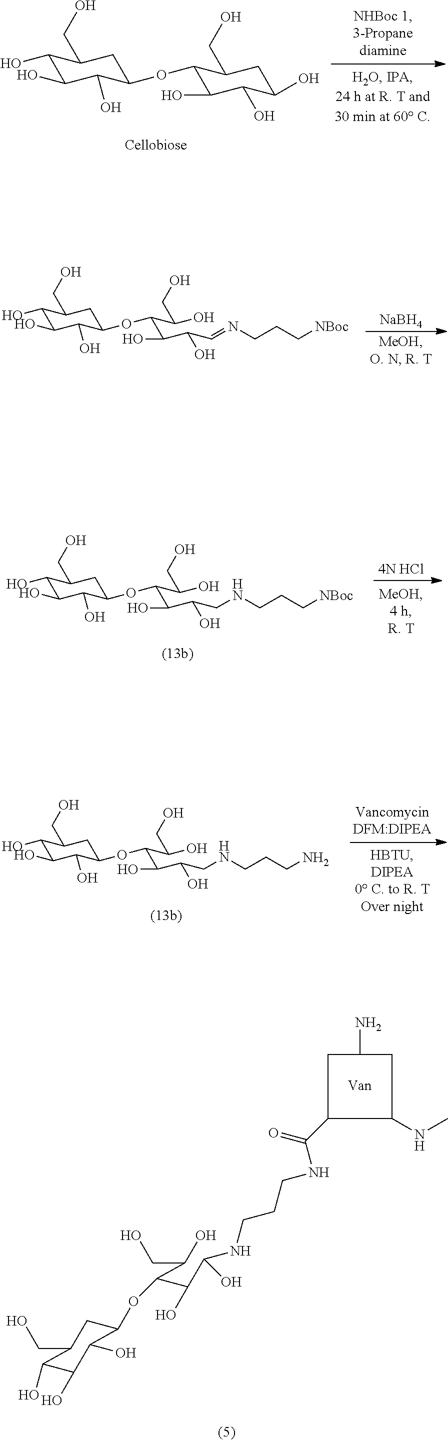

### Synthesis of 13a:

About 1 g of cellobiose was dissolved in 6 mL of millipore water. Then 0.85 g of (1.2 equivalents) of N-Boc-1,3-propanediamine was dissolved separately in 10 mL of isopropanol and added to the solution of cellobiose drop wise. The reaction mixture was stirred at room temperature for 24 h, then at 60° C. for 30 minutes. Now the solvent was evaporated to dryness and residue was washed with ethyl acetate and chloroform. Finally the remained solid was dried by high vacuum pump. This residue (1.4 g) was dissolved in 5 mL of dry methanol and 0.14 g (1.4 equivalents) of sodium borohydride was added to it. The reaction mixture was stirred at room temperature for 12 h. The reaction mixture was filtered and the filtrate was evaporated to get the pure 13a (90%). FT-IR (NaCl): 3362 cm⁻¹ (—OH str.), 2930 cm⁻¹ (—CH₂— asym. str.), 2881 cm⁻¹ (—CH₂— sym. str.), 1690 cm⁻¹ (—NHBoc C═O str.). ¹H-NMR (400 MHz, DMSO-d6) δ/ppm: 4.298 (d, 1H), 4.065-3.413 (m, 12H), 3.014 (m, 6H), 1.630 (m, 2H), 1.375 (s, 9H). ¹³C-NMR (100 MHz, DMSO-d6) δ/ppm: 170.78, 102.88, 76.78, 71.23, 71.12, 70.42, 44.22, 43.98, 36.24, 23.56, 20.66. HRMS: m/z 501.2653 (observed); 501.2659 (calculated for M+H⁺).

### Synthesis of 13b:

About 1.3 g of 13a was dissolved in 3 mL of methanol, then 5 mL of 4N HCl was added to it. The reaction was stirred at ambient temperature for 4 h. Now the MeOH was removed from the reaction mixture and work up was done with chloroform and water. The aqueous layer was collected and dried by using lyophilizer to get the pure 13b (75%). FT-IR (NaCl): 3329 cm⁻¹ (—OH and —NH₂ sym., asym. str.), 2929 cm⁻¹ (—CH₂— asym. str.), 2885 cm⁻¹ (—CH₂— sym. str.). ¹H-NMR (400 MHz, DMSO-d6) δ/ppm: 4.452 (d, 1H), 4.072, 3.602, 3.598, 3.421, (m, 12H), 3.025 (m, 6H), 1.651 (m, 2H). ¹³C-NMR (100 MHz, DMSO-d6) δ/ppm: 102.32, 76.91, 71.36, 71.10, 70.27, 44.26, 44.17, 36.20, 23.56. HRMS: m/z 401.2159 (observed); 401.2135 (calculated for M+H⁺).

### Synthesis of 5:

Vancomycin hydrochloride (100 mg, 67 μmol) was dissolved in 1:1 mixture of dry dimethyl formamide (1 mL) and dry dimethyl sulfoxide (1 mL). To this mixture, two equivalents of 13b in 1 mL of dry dimethylformamide was added. The reaction mixture was cooled to about 0° C., and about 0.22 mL (1.5 equivalents) of 0.45 M benzotriazole-N,N,N′,N′-tetramethyl-uronium-hexafluorophosphate (HBTU) solution in DMF was added followed by about 58 μL (5.0 equivalents) of diisopropylethylamine (DIPEA). The reaction mixture was then allowed to warm to room temperature and stirred for about 8-12 h. The product was purified by preparative reversed-phase HPLC using about 0.1% trifluoro-acetic acid in H₂O/acetonitrile mixture and then lyophilized to afford tris-(trifluoroacetate) salts of final compounds (50-55 μmol, 75-80%). These vancomycin-sugar conjugates were purified and characterized by ¹H-NMR and HR-MS (Table 1). The purification was done by preparative reverse phase HPLC using 0.1% trifluoro acetic acid (TFA) in water/acetonitrile (0-100%) as mobile phase. C18 column (10 mm diameter, 250 mm length) and UV detector (at 270 nm wave length) were used. The collected fractions, from HPLC were frozen by liquid N₂ and lyophilized in freeze dryer.

### Example 6

### Preparation of 6

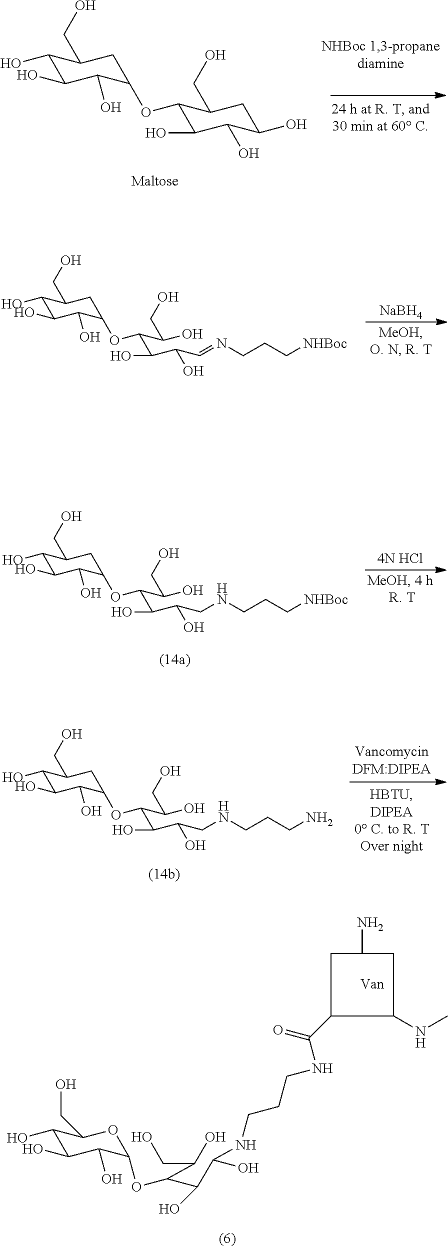

### Synthesis of 14a:

About 1 g of maltose was dissolved in 6 mL of millipore water. Then 0.85 g (1.2 equivalents) of N-Boc-1,3-propanediamine was dissolved separately in 10 mL of isopropanol and added to the solution of maltose drop wise. The reaction mixture was kept at ambient temperature for 24 h, then at 60° C. for 30 minutes. Now the solvent was evaporated to dryness and residue was washed with ethyl acetate and chloroform. Finally the remained solid was dried by high vacuum pump. This residue (1.4 g) was dissolved in 5 mL of dry methanol and 0.14 g (1.4 equivalents) of sodium borohydride was added to it. The reaction mixture was stirred at room temperature for 12 h. Then the reaction mixture was filtered and the filtrate was evaporated to get the pure 14a (86%). FT-IR (NaCl): 3354 cm⁻¹ (—OH str., —NH— sym., asym. str.), 2927 cm⁻¹ (—CH₂— asym. str), 2821 cm⁻¹ (—CH₂— sym. str.), 1690 cm⁻¹ (—NHBoc C═O str.). ¹H-NMR (400 MHz, DMSO-d6) δ/ppm: 4.815 (d, 1H), 4.407-3.388 (m, 12H), 3.102-2.669 (m, 6H), 1.630 (t, 2H), 1.378 (s, 9H), ¹³C-NMR (100 MHz, DMSO-d6) δ/ppm: 171.45, 103.15, 77.23, 70.85, 70.12, 68.67, 48.87, 44.54, 36.98, 23.87, 21.12. HRMS: m/z 501.2657 (observed); 501.2659 (calculated for M+H⁺).

### Synthesis of 14b:

About 1.2 g of 14a was dissolved in 3 mL of methanol, then 5 mL of 4N HCl was added to it. The reaction was kept at room temperature for 4 h. Now the methanol was removed from the reaction mixture and work up was done with chloroform and water. The aqueous layer was collected and dried by lyophilizer to get the pure 14b (80%). FT-IR (NaCl): 3339 cm⁻¹ (—OH and —NH₂ sym., asym. str.), 2928 cm⁻¹ (—CH₂— asym. str.) 2886 cm⁻¹ (—CH₂— sym. str.). ¹H-NMR (400 MHz, DMSO-d6) δ/ppm: 5.405 (d, 1H), 4.734-3.442 (m, 12H), 3.041-2.879 (m, 6H), 1.960 (t, 2H). ¹³C-NMR (100 MHz, DMSO-d6) δ/ppm: 103.05, 76.52, 71.35, 70.25, 68.48, 49.52, 44.24, 36.18, 23.55. HRMS: m/z 401.2143 (observed); 401.2135 (calculated for M+H⁺).

### Synthesis of 6:

Vancomycin hydrochloride (100 mg, 67 μmol) was dissolved in 1:1 mixture of dry dimethyl formamide (1 mL) and dry dimethyl sulfoxide (1 mL). To this mixture, two equivalents of 14b in 1 mL of dry dimethylformamide was added. The reaction mixture was cooled to about 0° C., and about 0.22 mL (1.5 equivalents) of 0.45 M benzotriazole-N,N,N′,N′-tetramethyl-uronium-hexafluorophosphate (HBTU) solution in DMF was added followed by about 58 μL (5.0 equivalents) of diisopropylethylamine (DIPEA). The reaction mixture was then allowed to warm to room temperature and stirred for about 8-12 h. The product was purified by preparative reversed-phase HPLC using about 0.1% trifluoro acetic acid in H₂O/acetonitrile mixture and then lyophilized to afford tris-(trifluoroacetate) salts of final compounds (50-55 μmol, 75-80%). These vancomycin-sugar conjugates were purified and characterized by ¹H-NMR and HR-MS (Table 1). The purification was done by preparative reverse phase HPLC using 0.1% trifluoro acetic acid (TFA) in water/acetonitrile (0-100%) as mobile phase. C18 column (10 mm diameter, 250 mm length) and UV detector (at 270 nm wave length) were used. The collected fractions, from HPLC were frozen by liquid N₂ and lyophilized in freeze dryer.

### Example 7

### Preparation of 7

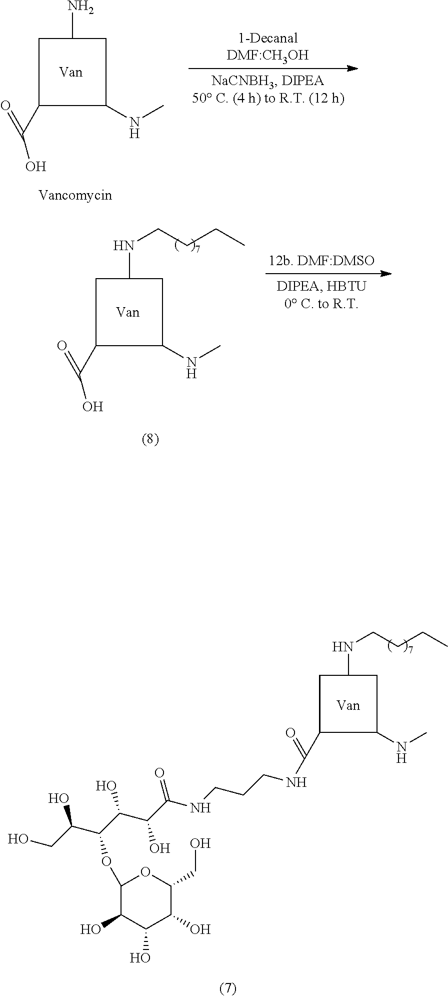

### Synthesis of 8:

Diisopropylethylamine (46 μL, 2.0 equivalents) was added to a solution of vancomycin hydrochloride (250 mg, 1.0 equivalent, 167.5 μmol) in 1:1 mixture of dry dimethylformamide (2 mL) and dry methanol (2 mL). About 30 μL (1.2 equivalents) of 1-decanal was added to the reaction mixture. Then the solution was heated at 50° C. for 2 h and then allowed to cool to room temperature prior to addition of sodium cyanoborohydride (20 mg, 2.0 equivalents). The reaction mixture was then stirred at 50° C. for additional 2 h and allowed to cool to ambient temperature for overnight. The product was purified by preparative reversed-phase HPLC using about 0.1% trifluoro acetic acid in H2O/acetonitrile mixture and then lyophilized to afford trifluoroacetate salt of compound 8 (75-80%). The purification was done by preparative reverse phase HPLC using 0.1% trifluoro acetic acid (TFA) in water/acetonitrile (0-100%) as mobile phase. C18 column (10 mm diameter, 250 mm length) and UV detector (at 270 nm wave length) were used. The collected fraction, from HPLC was frozen by liquid N, and lyophilized in freeze dryer.

### Synthesis of 7:

Compound 8 (100 mg, 67 μmol) was dissolved in 1:1 mixture of dry dimethyl formamide (1 mL) and dry dimethyl sulfoxide (1 mL). To this two equivalents of 12b in 1 mL of dry dimethylformamide was added. The reaction mixture was cooled to about 0° C., and about 0.22 mL (1.5 equivalents) of 0.45 M HBTU solution in DMF was added followed by about 58 μL (5.0 equivalents) of diisopropylethylamine (DIPEA). The reaction mixture was then allowed to warm to room temperature and stirred for about 8-12 h. The product was purified by preparative reversed-phase HPLC using about 0.1% trifluoro acetic acid in H₂O/acetonitrile mixture and then lyophilized to afford tris-(trifluoroacetate) salts of final compounds (50-55 μmol, 75-80%).

### Example 8

### Preparation of 15, 16, 17, and 18

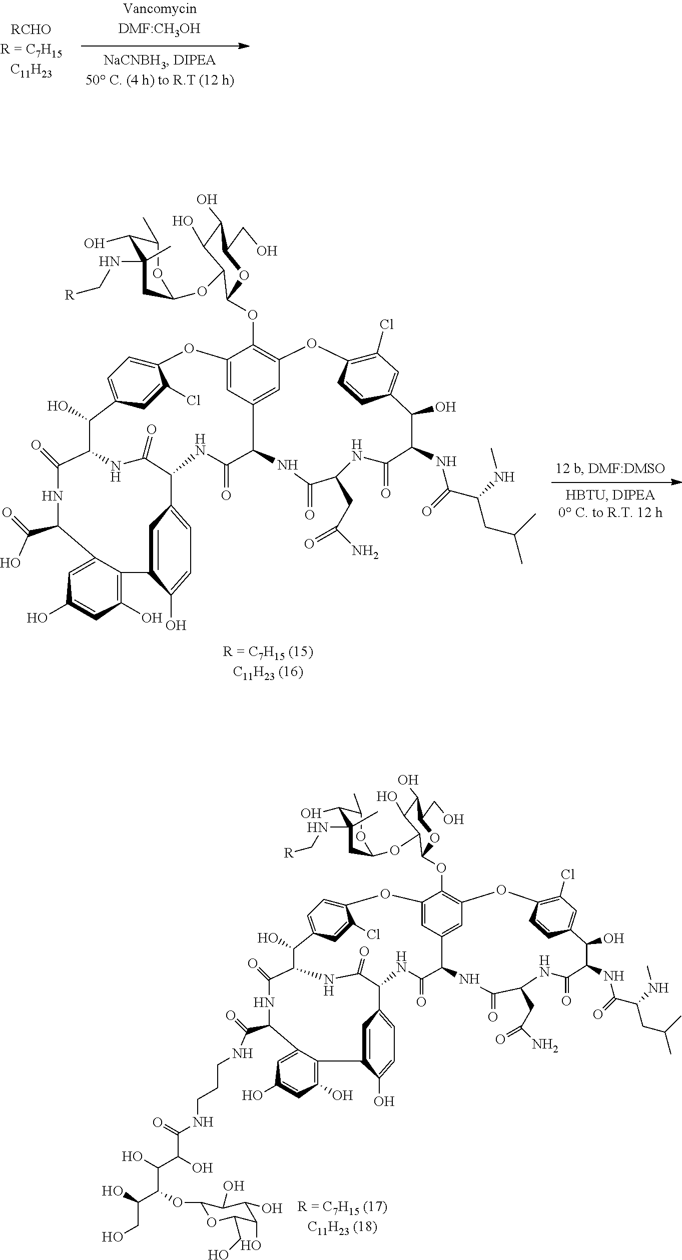

### Synthesis of 15 and 16:

Vancomycin hydrochloride (about 150 mg) was dissolved in dry dimethyl formamide (1 mL) and dry methanol (I mL). To this one equivalent of 1-octanal or 1-dodecanal and 1.2 equivalents of diisopropylethylamine (DIPEA) were added. The reaction mixture was stirred at 50° C. for 2 h and then allowed to cool to room temperature prior to addition of sodium cyanoborohydride (2.0 equivalents). Then, the reaction mixture was stirred at 50° C. for additional 2 h and allowed to cool to ambient temperature for overnight. The product was purified by preparative reversed-phase HPLC using 0.1% trifluoro acetic acid in H₂O/acetonitrile mixture and then lyophilized to afford trifluoroacetate salt compound 15 or 16 in 75-77% yield.

Compound 15: ¹H NMR (400 MHz, DMSO-d₆) δ 9.44 (s, 1H), 9.18 (s, 1H), 9.08 (s, 1H), 8.98 (bs, 1H), 8.88 (bs, 1H), 8.71-8.51 (m, 2H), 8.09 (bs, 1H), 7.81 (bs, 2H), 7.59-7.45 (m, 4H), 7.31-7.1 (m, 3H), 6.78-6.67 (m, 2H), 6.35-6.24 (dd, 2H), 6.0-5.93 (m, 2H), 5.75-5.65 (m, 2H), 5.36-5.2 (m, 6H), 4.91-4.90 (d, 1H), 4.61-4.42 (m, 4H), 4.18-4.08 (m, 4H), 2.67-2.61 (m, 3H), 1.80-1.75 (m, 1H), 1.66-1.51 (m, 4H), 1.24 (m, 13H), 1.09-1.07 (d, 3H), 0.91-0.85 (m, 10H).

Compound 16: ¹H NMR (400 MHz. DMSO-d₆) δ 9.41 (s, 1H), 9.20 (s, 1H), 9.12 (s, 1H), 9.01 (bs, 1H), 8.88 (bs, 1H), 8.69-8.53 (m, 2H), 8.25 (bs, 1H), 7.93 (bs, 2H), 7.61-7.45 (m, 4H), 7.33-7.21 (m, 3H), 6.78-6.67 (m, 2H), 6.38-6.24 (dd, 2H), 5.99-5.85 (m, 2H), 5.83-5.63 (m, 2H), 5.36-5.2 (m, 6H), 4.95-4.93 (d, 1H), 4.53-4.42 (m, 4H), 4.21-4.10 (m, 4H), 2.71-2.61 (m, 3H), 1.80-1.77 (m, 1H), 1.66-1.55 (m, 4H), 1.28 (m, 21H), 1.09-1.07 (d, 3H), 0.91-0.86 (m, 10H).

### Synthesis of 17 and 18:

Compound 15 or 16 (67 μmol) was dissolved in dry dimethyl formamide (1 mL) dry dimethyl sulfoxide (1 mL). To this, two equivalents of compound 12b in 1 mL of dry dimethylformamide was added. The reaction mixture was cooled to 0° C., and 0.22 mL (1.5 equivalents) of 0.45 M HBTU solution in DMF was added followed by 58 μL of DIPEA (5.0 equivalents). The reaction mixture was then allowed to warm to room temperature and stirred for 8-12 h. The products were purified by preparative reversed-phase HPLC to more than 95% using 0.1% trifluoro acetic acid in H₂O/acetonitrile mixture and then lyophilized to afford tris-(trifluoroacetate) salts of final compounds (47-54 μmol, 70-80%).

Compound 17: ¹H NMR (400 MHz, DMSO-d₆) δ 9.33 (s, 1H), 9.03-8.99 (d, 2H), 8.69 (bs, 1H), 8.48-8.46 (d, 2H), 8.14-8.06 (m, 2H), 7.84-7.39 (m, 9H), 7.35-7.06 (m, 4H), 6.78-6.66 (m, 2H), 6.48 (bs, 1H), 6.37-6.22 (dd, 2H), 5.90-5.62 (m, 5H), 5.36-5.10 (m, 8H), 4.91 (bs, 1H), 4.61-4.60 (d, 2H), 4.46-4.45 (d, 2H), 4.37-4.35 (d, 2H), 4.24-4.22 (d, 3H), 4.11-4.08 (t, 3H), 2.79-2.78 (d, 2H), 2.70-2.66 (m, 2H), 2.33-2.31 (m, 2H), 2.19 (bs, 1H), 2.00-1.97 (m, 1H), 1.80-1.65 (m, 5H), 1.59-1.53 (m, 3H), 1.36 (s, 3H), 1.25 (m, 13H), 1.10-1.08 (d, 3H), 0.92-0.84 (m, 10H).

Compound 18: ¹H NMR (400 MHz, DMSO-d₆) δ 9.33 (s, 1H), 9.04-8.99 (d, 2H), 8.69 (bs, 1H), 8.48-8.47 (d, 2H), 8.14-8.05 (m, 2H), 7.84 (s, 2H), 7.67 (bs, 3H), 7.54-7.45 (m, 4H), 7.30-7.21 (m, 3H), 7.07 (bs, 1H), 6.78-6.69 (m, 3H), 6.37-6.22 (dd, 2H), 5.92 (bs, 2H), 5.80-5.75 (m, 3H), 5.63-5.62 (d, 2H), 5.36-5.10 (m, 7H), 4.91-4.90 (d, 1H), 4.61-4.60 (d, 2H), 4.46-4.45 (d, 2H), 4.37-4.35 (d, 2H), 4.24-4.20 (m, 2H), 4.12-4.09 (t, 2H), 3.71-3.66 (m, 4H), 2.81-2.78 (m, 3H), 2.67-2.66 (m, 1H), 2.33-2.32 (m, 2H), 2.00-1.97 (d, 1H), 1.80-1.64 (m, 4H), 1.58-1.53 (m, 3H), 1.36 (s, 3H), 1.24 (m, 21H), 1.09-1.08 (d, 3H), 0.92-0.83 (m, 10H).

These vancomycin-sugar conjugates were purified and characterized by ¹H-NMR and HR-MS (Table 1). The purification was done by preparative reverse phase HPLC using 0.1% trifluoro acetic acid (TFA) in water/acetonitrile (0-100%) as mobile phase. C18 column (10 mm diameter, 250 mm length) and UV detector (at 270 nm wave length) were used. The collected fractions, from HPLC were frozen by liquid N₂ and lyophilized in freeze dryer.

TABLE 1
Characterization of vancomycin-sugar conjugates

| | | Molecular weight | Molecular weight |
|---|---|---|---|
| | Retention Time | (cal) | (obs. by HR-MS) |
| | (HPLC) | [daltons] | [daltons] |
| Compound | [minutes] | {[M + 2H]²⁺/2} | {[M + 2H]²⁺/2} |
| Vancomycin | 7.934 | 725.6253 | 724.7177 |
| 1 | 7.505 | 828.2311 | 827.2645 |
| 2 | 7.474 | 828.2311 | 828.2641 |
| 3 | 7.286 | 842.7497 | 842.2764 |
| 4 | 7.273 | 923.8198 | 823.8035 |
| 5 | 7.182 | 916.8285 | 916.8133 |
| 6 | 7.118 | 916.8285 | 916.8015 |
| 7 | 11.4 | 993.9523 | 993.8801 |
| 8 | 12.003 | 795.757 | 795.798 |
| 15 | 11.003 | 780.735 | 780.719 |
| 16 | 13.8 | 808.785 | 808.79 |
| 17 | 10.5 | 978.931 | 978.952 |
| 18 | 13.1 | 1006.981 | 1006.99 |

### Example 9

### In-Vitro Antibacterial Activity

### Minimum Inhibitory Concentration (MIC):

All test compounds were assayed in a micro-dilution broth format. Stock solutions were made by serially diluting the compounds using autoclaved millipore water or broth media. The antibacterial activity of the compounds was determined against methicillin-sensitive S. aureus (MSSA), methicillin-resistant S. aureus (MRSA), vancomycin-intermediate-resistant S. aureus (VISA), vancomycin-sensitive E. faecium (VSE) and vancomycin-resistant E. faecium (VRE). Bacteria, to be tested, were grown for about 10 h in the suitable media, MSSA, MRSA and VISA were grown in yeast-dextrose broth (about 1 g of beef extract, about 2 g of yeast extract, about 5 g of peptone and about 5 g of NaCl in about 1000 mL of sterile distilled water (pH-7)). For solid media, about 5% agar was used along with above mentioned composition. VSE and VRE were cultured in brain heart infusion broth (Himedia). The bacterial samples were freeze dried and stored at −80° C. About 5 μL of these stocks were added to about 3 mL of the nutrient broth and the culture was grown for about 6 h at about 37° C. prior to the experiments. This 6 h grown culture gives about 10⁹ cfu/mL and this was determined by spread plating method. The 6 h grown culture was diluted to give effective cell concentration of about 10⁵ cfu/mL which was then used for determining MIC. Compounds were serially diluted, in sterile water (2 fold dilution is employed) in a way that the working concentration was about 10 μM for MSSA, MRSA, and VSE while for VRE and VISA it was about 100 μM. About 50 μL of these serial dilutions were added to the wells of 96 well plate followed by the addition of about 150 μL of bacterial solution. The plates were then incubated at about 37° C., 150 rpm in the incubator and O.D at 600 nm was recorded at an interval of about 10 h and 24 h using TECAN (Infinite series, M200 pro) Plate Reader. Each concentration had triplicate values and the whole experiment was done at least twice and the MIC value was determined by taking the average of triplicate. O. D. values for each concentration and plotting it against concentration. The data was then subjected to sigmoidal fitting. From the curve the MIC value was determined, as the point in the curve where the O. D. was similar to that of control having no bacteria.

The antibacterial activities of compounds 1 to 8, 15 to 18, and vancomycin against Staphylococci (MSSA, MRSA and VISA) and Enterococci (VSE and VRE) were summarized in Table 2. The antibacterial activities of these derivatives were seen to be dependent on the nature of sugar moiety whether cyclic or acyclic. In case of wild type bacterial strains MSSA, the antibacterial activity varied from 0.3 to 1.4 μM while for VSE it was about 0.4 to 1.7 μM. Amongst these, the derivative 6 bearing cyclic and acyclic form of sugar moiety showed the best activity against both MSSA and VSE. Further, most exciting results were obtained in case of resistant bacteria. When tested against highly pathogenic MRSA and VISA, these derivatives exhibited minimum inhibitory concentration (MIC) in the range 0.3 to 1.7 μM and 0.2 to 2.4 μM respectively. Again the derivative 6 showed MIC of 0.3 μM against both MRSA and VISA implying about 2 fold and 40 fold more active than vancomycin respectively. Derivative 7 showed about 65 fold more active than vancomycin with the lowest MIC value of 0.2 μM against VISA. Considering VRE (VanA phenotype), the MIC fell in the range of 1.0 to >100 μM. The derivative 7 has showed >700 fold higher activity than vancomycin. Also, these compounds showed good activity against clinical isolates of methicillin-resistant bacteria (Table 3).

TABLE 2
Antibacterial activities of vancomycin-sugar conjugates.
^{a}Methicillin-sensitive S. aureus (MTCC 737).
^{b}Methicillin-resistant S. aureus (ATCC 33591). ^{c}Vancomycin
intermediate resistant S. aureus. ^{d}Vancomycin-sensitive
E. faecium (ATCC 19634). ^{e}Vancomycin-resistant
E. faecium (VanA, ATCC 51559), ^{f}Vancomycin-resistant
E. faecalis (VanA, ATCC 51575).
MIC (μM)

| | | | | | VRE | VRE |
|---|---|---|---|---|---|---|
| Compound | MSSA^{a} | MRSA^{b} | VISA^{c} | VSE^{d} | (VanA)^{e} | (VanB)f |
| Vancomycin | 0.63 | 0.63 | 13.0 | 0.6 | >700 | 250 |
| 1 | 1.4 | 1.2 | 2.4 | 0.6 | >100 | — |
| 2 | 1.2 | 1.4 | 2.02 | 1.2 | >100 | — |
| 3 | 0.6 | 0.7 | 0.88 | 0.5 | 54.0 | — |
| 4 | 0.3 | 0.38 | 0.3 | 0.4 | 36.0 | |
| 5 | 1.0 | 1.0 | 1.08 | 0.66 | >100 | — |
| 6 | 1.0 | 1.0 | 0.99 | 0.5 | >100 | — |
| 7 | 0.2 | 0.3 | 0.2 | 0.15 | 1.0 | 1.0 |
| 8 | 0.3 | 0.3 | 0.32 | 0.2 | 14.0 | 6.2 |
| 15 | 0.3 | 0.3 | 0.4 | 0.4 | 25.0 | 12.5 |
| 16 | 0.3 | 0.3 | 0.3 | 0.2 | 7.0 | 3.1 |
| 17 | 0.2 | 0.3 | 0.31 | 0.2 | 2.0 | 6.2 |
| 18 | 0.2 | 0.3 | 0.22 | 0.2 | 0.8 | 1.0 |

TABLE 3
In-vitro antibacterial activity against clinical
isolates of methicillin-resistant bacteria.
MIC (μM)

| Compound | S. epidermidis | S. haemolyticus | S. aureus |
|---|---|---|---|
| Vancomycin | 0.9 | 1.4 | 0.7 |
| 17 | 0.3 | 0.4 | 0.2 |
| 7 | 0.3 | 0.41 | 0.3 |
| 18 | 0.35 | 0.5 | 0.3 |

### Example 10

### Ex-Vivo Whole Blood Assay

Ex-vivo whole blood assay was performed to compare the abilities of these compounds to retain activity in complex media. To 30 μL of VISA in saline (0.9% NaCl; 10⁶ CFU/mL) 10 μL of test compounds (vancomycin and compound 7) and 270 μL of fresh human whole blood were added and incubated, at 37° C. for about 3 h. After the incubation period, antibacterial activity was determined by finding the bacterial titer in the infected blood.

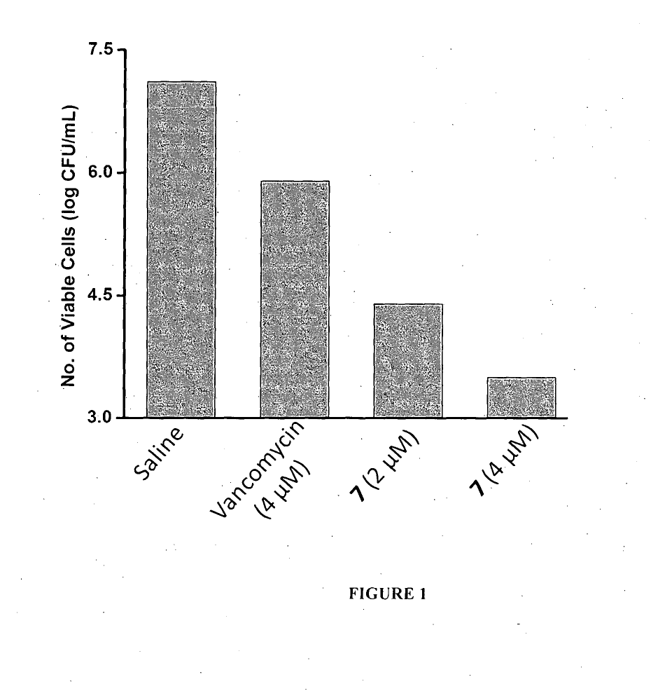

Compound 7 showed rapid bactericidal activity against VISA after incubation for 3 h in 90% human whole blood, whereas vancomycin showed slow killing (FIG. 1). This result indicates that these derivatives could maintain antibacterial activity in-vivo with nominal loss due to non-specific interactions with tissue components.

### Example 11

### In-Vivo Time Dependent Whole Blood Assay

The derivative 7 and vancomycin were administered in a single intravenous injection (0.2 mL saline) to normal pathogen-free, female CD-1 mice. Doses of 12 mg kg⁻¹ were administered to three mice per data point. At the specified time-points (0, 3, 6, 12, 24 and 48 h) mice were euthanized (using ether), blood samples were collected from the ocular puncture. 60 μL of VISA in saline (0.9% NaCl; 10⁶ CFU/mL) was added to 540 μL of whole blood which was drawn from the mice and incubated at 37° C. for 3 h. After the incubation period, antibacterial activity was determined by finding the bacterial titer in the infected blood.

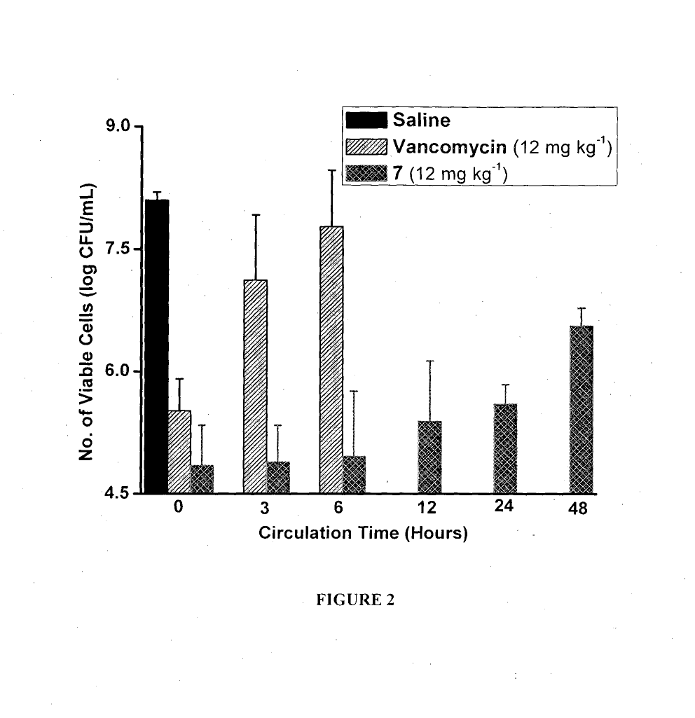

Compound 7 was found to be active even up to 24 h and showed 3-log₁₀ CFU/mL reduction, whereas vancomycin exhibited nominal activity at 3 h and did not show any activity at 6 h (FIG. 2). This study indicates that most of the vancomycin was cleared from the mice within 3 h, while the compound 7 persevered in the mice even after 24 h and showed antibacterial activity. This study indicates that compound 7 can have improved pharmacological properties compared to parent compound, vancomycin.

### Example 12

### Time-Kill Assay

The bactericidal activity was assessed by the kinetics or the rate at which it affects the killing action of the compound. Briefly methicillin-resistant vancomycin-intermediate S. aureus (MR-VISA) grown in Yeast-Dextrose broth. A starting inoculum of 1.6×108 CFU/ml was used as initial bacterial colony count. Vancomycin and compound 7 having final concentrations of 2 μM and 4 μM were inoculated with MR-VISA suspensions having starting inocula of 1.6×108 CFU/ml. Bacterial suspension containing specified concentrations of the compound along with negative control (which contains only 0.9% Saline) was incubated at 37° C. with shaking. Aliquots (20 μl) were removed from the cultures at different time intervals and were serially diluted 10-fold in 0.9% saline and plated onto sterile Yeast-Dextrose agar medium. The number of viable cells was determined by plating the 10-fold serial dilution of each sample onto Yeast-dextrose agar medium. Plates were then incubated for 24 h at 37° C., CFU was counted and the total bacterial log 10 CFU/ml was determined.

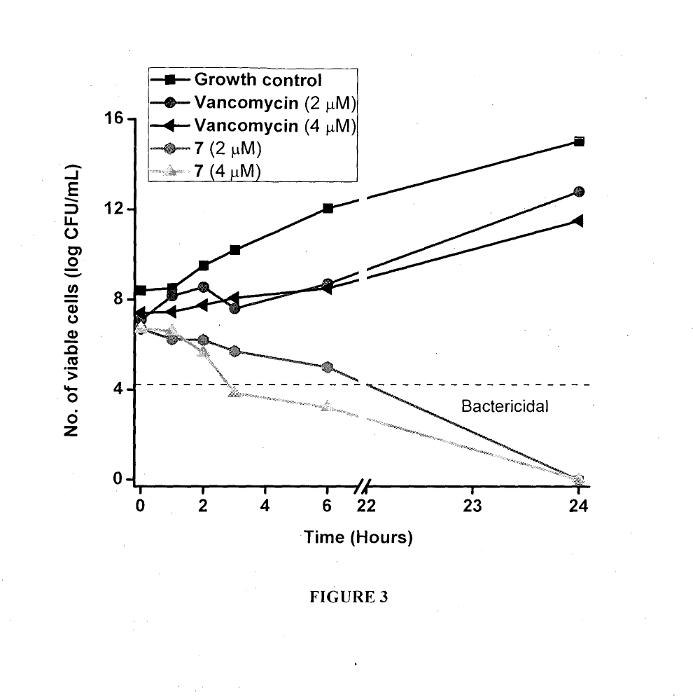

FIG. 3 exhibits in-vitro time time-kill kinetics of vancomycin-sugar conjugate. All points below the dotted line in FIG. 3 indicate >3 log₁₀ CFU/mL reduction. Vancomycin showed relatively slow killing or bacteriostatic effect and did not appear to be dose dependent, whereas killing by compound 7 was rapid and the rate of killing increased with the concentration, where we found 4- to 5-log 10-CFU/ml reduction at 3 h for the concentration 4 μM.

### Example 13

### Methicillin-Resistant Vancomycin Intermediate Staphylococcus aureus (MR-VISA) Infection

### In-Vivo Antibacterial Activity:

About six-week-old, female CD-1 mice (weight, ˜19-24 g) were used for the experiments. The mice were rendered neutropenic (˜100 neutrophils/ml) by injecting two doses of cyclophosphamide intraperitoneally 4 days (150 mg/kg) and 1 day (100 mg/kg) before the infection experiment. 50 μl of ˜10⁷ CFU/ml concentration of the bacterial inoculum (MR-VISA) was injected into the thigh. One hour after inoculation, animals were treated intravenously with saline, vancomycin, linezolid and compound 7 at 12 mg/kg and 24 mg/kg of body weight (24 h total dosage). At 24 h post first treatment, cohorts of animals were euthanized (using ether) and the thighs were collected aseptically. The thigh was weighed (0.7 g-0.9 g) and placed into 10 ml of sterile saline and homogenized. The dilutions of the homogenate were plated onto agar plates, which were incubated overnight at 37° C. The bacterial titer was expressed as log₁₀ CFU/g of thigh weight.

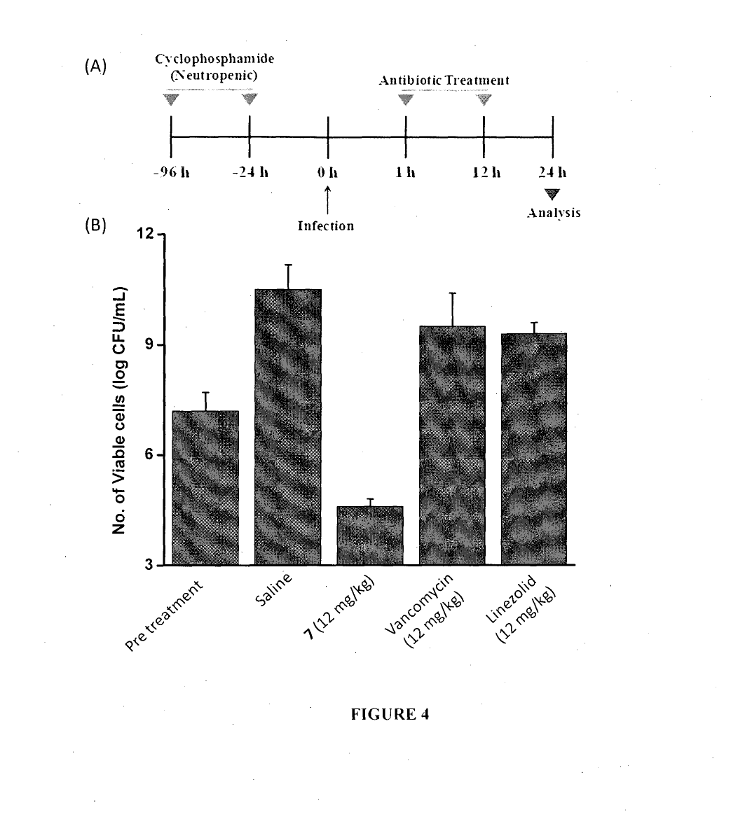

The experimental design for in-vivo activity of compound 7 in comparison with vancomycin and linezolid against MR-VISA (n=5) is shown in FIG. 4A. Data are expressed as means±SD (error bars). The in-vivo efficacy of compound 7 in comparison with linezolid and vancomycin against MR-VISA was shown in FIG. 4B. The bacterial density taken from control animals prior to initiation of dosing was determined to be 7.1+0.28 log₁₀ CFU/g. After 24 h of the initial treatment, antibacterial activity was determined by finding the bacterial titer in the infected thighs. Vancomycin and linezolid produced 50% maximal response from the vehicle treated mice (ED₅₀). In contrast, compound 7 showed excellent efficacy, where it produced ˜3.0 log₁₀ CFU/g reduction in bacterial count from the initial titer (ED_{3-log kill}) at 12 mg/kg.

### Pharmacodynamics Against MR-VISA Infection:

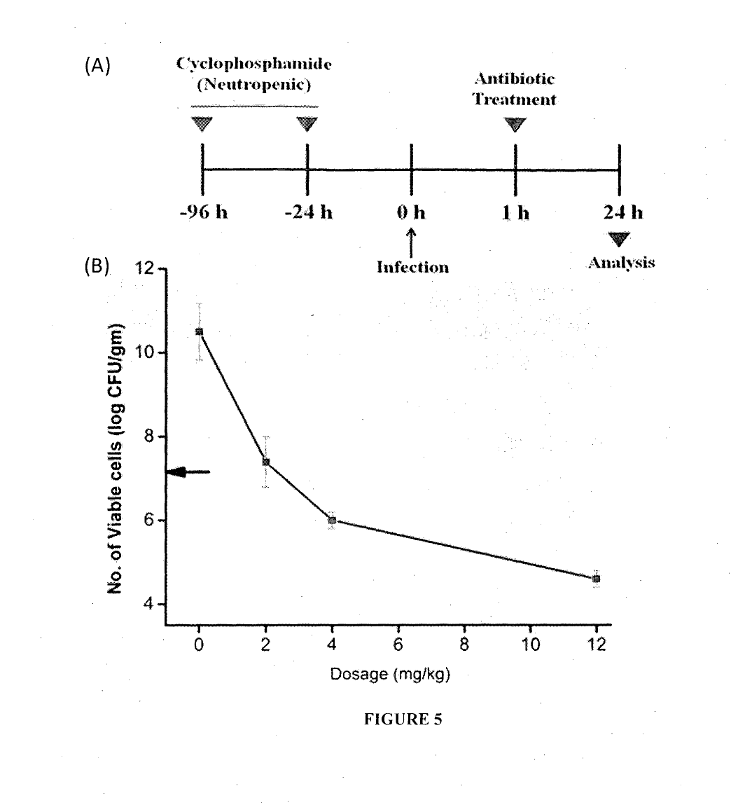

The experimental design for pharmacodynamics of compound 7 in comparison against MR-VISA (n=5) is shown in FIG. 5A. Data are expressed as means±SD (error bars). A separate single-dose study of compound 7 was performed in neutropenic mice inoculated in the thigh with 50 μL of MR-VISA (10⁷ CFU/ml). Infected animals were treated intravenously, at 1 h post infection, with 2 mg/kg, 4 mg/kg, 8 mg/kg and 12 mg/kg. At 24 h post inoculation mice were sacrificed and the thigh tissues were harvested for determination of bacterial titer as mentioned above.

The pretreatment bacterial titer in the thigh was 7.2±0.2 log₁₀ CFU/g. In vehicle treated controls, thigh titer increased to 10.3±0.1 log₁₀ CFU/g within 24 h. Compound 7 produced comparable dose dependent reductions in the bacterial titer at each of four dosing regimens (FIG. 5B). The single compound 7 dose that resulted in 50% maximal bacterial killing (ED₅₀) was 1.05 mg/kg (Table 4). The compound 7 dose that resulted in a 24-h colony count similar to the pretreatment count was 2.22 mg/kg (EDₛₜₐₛᵢₛ). The value of 1-log₁₀ kill dose (ED_{1-log kill}) for compound 7 was 3.7 mg/kg. It was found that at the highest dosing regimen (12 mg/kg) compound 7 showed ED_{2.6-log kill} (FIG. 5B).

TABLE 4
Point dose estimates required to achieve different
pharmacodynamic end points against MR-VISA
(Methicillin-resistant Vancomycin intermediate S. aureus) thigh
infection model
Pharmacodynamic end points (mg/kg)

| Bacterial | | | | | | |
|---|---|---|---|---|---|---|
| strain | Drug | ED₅₀ | EDₛₜₐₛᵢₛ | ED_{1-log kill} | ED_{2-log kill} | ED_{2.6-log kill} |
| MR-VISA | Com- | 1.0 | 2.2 | 3.7 | 8.8 | 12 |
| (Pretreatment | pound 7 | | | | | |
| 7.2 log₁₀ | | | | | | |
| CFU/g) | | | | | | |

### Example 14

### Pharmacokinetics

A single dose pharmacokinetic analysis of compound 7 was performed in CD-1 female mice. Mice were administered a single intravenous dose of 12 mg/kg. Blood samples were collected from mice by retro-orbital aspiration and placed into heparinized tubes at different time intervals after dosing. The plasma was separated by centrifugation, and drug plasma concentrations were measured by microbiologic assay with Bacillus subtilis as the test organism. The lower limit of detection of the assay was 0.6 μg/ml. Pharmacokinetic parameters, including half-life, AUC and Cₘₐₓ were calculated by using non-compartmental model. The AUC was estimated up to 24 h and half-life (t_{1/2}) was calculated.

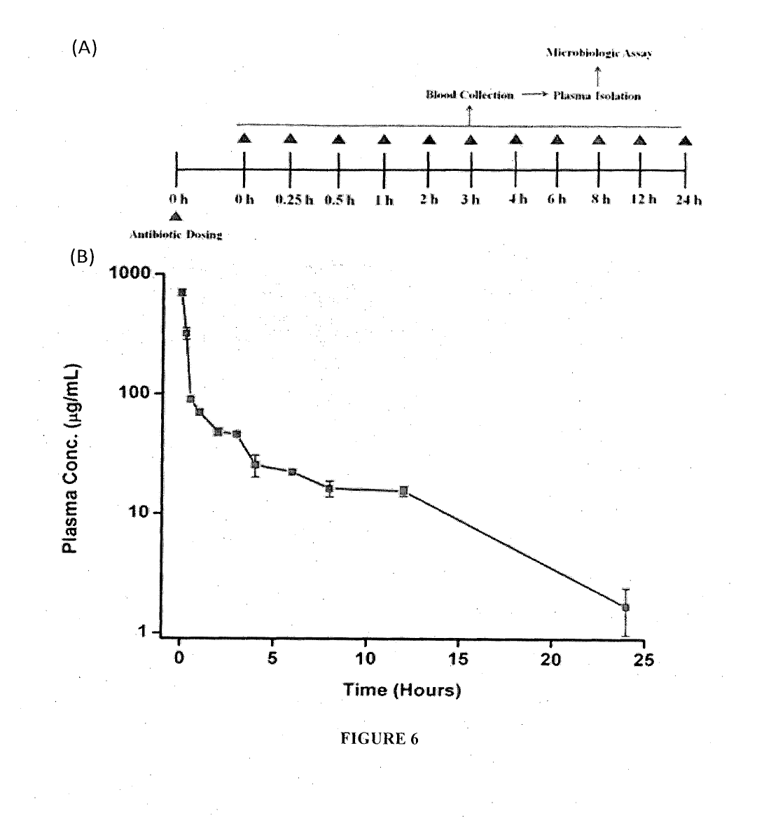

The experimental design for determining the pharmacokinetics profile of compounds of the present disclosure is shown in FIG. 6A. The abscissa shows the time, and the ordinate shows the plasma drug concentration (n=5 per group). Data are expressed as means±SD (error bars). The Pharmacokinetics of i.v. administered compound 7 in mice is shown in FIG. 6B and Table 5. The compound demonstrates increased exposure as measured by area under concentration curve in mice. Time-concentration profiles of plasma for compound 7 are presented in FIG. 6B. Peak concentration in plasma was found to be 702.9 μg/ml. The AUC value in plasma, calculated from 0.083 h to 24 h was 562.4 μg·h/ml. The plasma half-life (t_{1/2}) of compound 7 was found to be 2.76 h with the clearance rate of 0.25 L/h/Kg.

TABLE 5
Single-dose pharmacokinetic parameters of compound 7 at 12 mg/kg
Pharmacokinetics parameters

| | Cₘₐₓ | Cₘᵢₙ | AUC_{0-24 h} | t_{1/2} | Clearance |
|---|---|---|---|---|---|
| Drug | (μg/ml) | (μg/ml) | (μg/ml/h) | (h) | (L/h/kg) |
| Compound 7 | 703 | 1.7 | 562 | 2.76 | 0.25 |

### Example 15

### In-Vivo Toxicology

### Systemic Toxicity:

Systemic toxicity was examined after i.v injection of compound 7 to CD-1 female mice. Each mouse was injected with a 0.2 ml of freshly prepared compound 7 solution in saline. The doses of the compound administered per group were according to OECD guidelines (OECD, 2005). Animals were directly inspected for adverse effects for 4 h, and mortality was observed for 14 days, thereafter, LD₅₀ was determined using Spearman-Karber method.

The in-vivo systemic toxicity of compound 7 after single-dose intravenous (i.v.) administration to mice and the LD50 value was found to be >100 mg/kg.

### Acute Toxicity:

For the evaluation of the acute toxicity, two groups of 10 mice each received intravenous injection of compound 7 at 12 mg/kg in 0.2 ml of sterilized saline. 10 mice were sacrificed at 48 h and the rest mice at 14 days to collect blood samples for analysis of biochemical parameters such as alanine transaminase (ALT), alkaline phosphatase (ALP), urea nitrogen, creatinine, sodium ion, potassium ion and chloride ion levels. Blood samples were analyzed at Gokula Metropolis clinical laboratory, Bengaluru, India. And also to examine the adverse effects of compound 7 in tissue level, we have isolated liver and kidney organs in 10% neutral formalin. Tissues were processed by dehydration in ascending grades of ethyl alcohol, clearing in xylol, embedding in paraffin wax and prepared sections of 5 μm thickness. Then paraffin sections were stained using haematoxylin and eosin, and observed under light microscope for histological changes.

The levels of the functional parameters of the liver and kidney and the concentrations of potassium and sodium ions were unchanged after 48 h and 14 days (Table 6). These studies indicate that Compound 7 did not cause any significant acute damage to liver and kidney functions, nor did it interfere with the balance of electrolytes in the blood. Gross anatomical and histopathological examination of liver and kidney sections from Compound 7 treated mice revealed no significant changes compared to control.

TABLE 6
Acute toxicology of compound 7.
Effect of Compound 7 on liver and kidney functions as well as balance of electrolytes in the blood

| | | | | Electrolytes in the blood | | |
|---|---|---|---|---|---|---|
| | | Urea | Kidney | | | Chloride |
| | Liver | Nitrogen | Creatinine | Potassium ion | Sodium ion | ion |
| Treatment | ALT (U L⁻¹) | (mg dL⁻¹) | (mg dL⁻¹) | (mmol L⁻¹) | (mmol L⁻¹) | (mmol L⁻¹) |
| Without | 60.27 ± 9.317 | 22.19 ± 3.24 | 0.32 ± 0.2 | 9.53 ± 1.45 | 143.75 ± 0.789 | 107.9 ± 1.91 |
| treatment | | | | | | |
| (Saline) | | | | | | |
| 48 h post- | 55.23 ± 5.24 | 19.52 ± 3.25 | 0.2 ± 0.133 | 6.86 ± 0.81 | 143.63 ± 1.65 | 112.9 ± 1.52 |
| treatment | P = 0.004 | P = 0.06 | P = 0.056 | P = 0.052 | P = 0.83 | P = 0.005 |
| | (<0.05) | (>0.05) | (>0.05) | (>0.05) | (>0.05) | (>0.05) |
| 14 days | 53.28 ± 3.78 | 24.46 ± 4.93 | 0.22 ± 0.1 | 6.75 ± 0.833 | 143.04 ± 0.71 | 110.85 ± 2.16 |
| post | P = 0.02 | P = 0.23 | P = 0.054 | P = 0.053 | P = 0.095 | P = 0.237 |
| treatment | (<0.05) | (>0.05) | (>0.05) | (>0.05) | (>0.05) | (>0.05) |
| Laboratory | 63-307 | 17-35 | 0.2-0.8 | 6.3-10 | 140-150 | 104-120 |
| range* | | | | | | |

Compound 7 causes no significant acute damage to the liver and kidney functions, nor does it interfere with the concentrations of potassium and sodium ions in the blood at a concentration of 12 mg/kg. The data are expressed as mean±standard deviation, based on values obtained from 10 mice (n=10). Statistical analysis was performed using Student's t-test. Differences are considered statistically significant with probability P<0.05. ALT, alanine transaminase; U, international unit.

### ADVANTAGE

The above mentioned implementation examples as described on this subject matter and its equivalent thereof have many advantages, including those which are described.

The disclosed compounds and/or derivatives in the present invention can provide better interaction with the cell wall of the bacteria through improved hydrogen bonding interactions. This increased association with bacterial cell wall precursors can serve as to inhibit the cell wall biosynthesis in both sensitive and resistant bacteria.

Although the subject matter has been described in considerable details with reference to certain preferred embodiments thereof, other embodiment are possible. As such, the spirit and scope of the appended claims should not be limited to the description of the preferred embodiments contained therein.

1. A compound of formula I 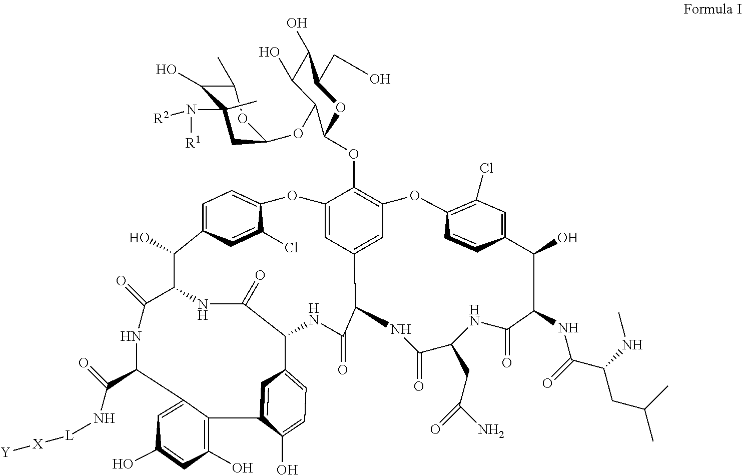
or its stereoisomers, prodrugs and pharmaceutically acceptable salts thereof:
wherein R¹ and R² are independently selected from the group consisting of hydrogen, a C₂-C₁₈ alkyl, a C₆-C₁₈ aryl, alkenyl, alkynyl, haloalkyl, arylalkyl, hydroxyalkyl, carboxyalkyl, cycloalkyl, cycloalkylalkyl, heterocyclyl, heterocyclylalkyl, heteroaryl; wherein alkyl, alkenyl, alkynyl, cycloalkyl, cycloalkylalkyl, arylalkyl, aryl, heteroaryl, heterocyclyl, and heterocyclylalkyl are independently unsubstituted or substituted with upto four substituents independently selected from alkyl, alkenyl, alkynyl, alkoxy, acyl, acyloxy, acylamino, amino, monoalkylamino, dialkylamino, trialkylamino, halogen, hydroxy, hydroxyalkyl, keto, thiocarbonyl, carboxy, alkylcarboxy, hydroxyamino, alkoxyamino, nitro, azido, cyano, cycloalkyl, cycloalkylalkyl, aryl, arylalkyl, heterocyclyl, heterocyclylalkyl, heteroaryl, heteroarylalkyl, cycloalkenyl, cycloalkylamino, arylamino, heterocyclylamino, heteroarylamino, cycloalkyloxy, aryloxy, heterocyclyloxy or heteroaryloxy;
L is a C₂-C₆ alkyl, a C₈-C₁₈ aryl, alkenyl, alkynyl, haloalkyl, arylalkyl, hydroxyalkyl, carboxyalkyl, cycloalkyl, cycloalkylalkyl, heterocyclyl, heterocyclylalkyl, heteroaryl; wherein alkyl, alkenyl, alkynyl, cycloalkyl, cycloalkylalkyl, arylalkyl, aryl, heteroaryl, heterocyclyl, and heterocyclylalkyl are independently unsubstituted or substituted with upto four substituents independently selected from alkyl, alkenyl, alkynyl, alkoxy, acyl, acyloxy, acylamino, amino, halogen, hydroxy, hydroxyalkyl, keto, thiocarbonyl, carboxy, alkylcarboxy, hydroxyamino, alkoxyamino, nitro, azido, cyano, cycloalkyl, cycloalkylalkyl, aryl, arylalkyl, heterocyclyl, heterocyclylalkyl, heteroaryl heteroarylalkyl, cycloalkenyl, cycloalkylamino, arylamino, heterocyclylamino, heteroarylamino, cycloalkyloxy, aryloxy, heterocyclyloxy or heteroaryloxy;
X is NH and O; and
Y is selected from the group consisting of cyclic monosaccharide, cyclic disaccharide, acyclic monosaccharide, acyclic disaccharide, and combinations thereof.

2. The compound as claimed in claim 1, wherein Y is selected from the group consisting of

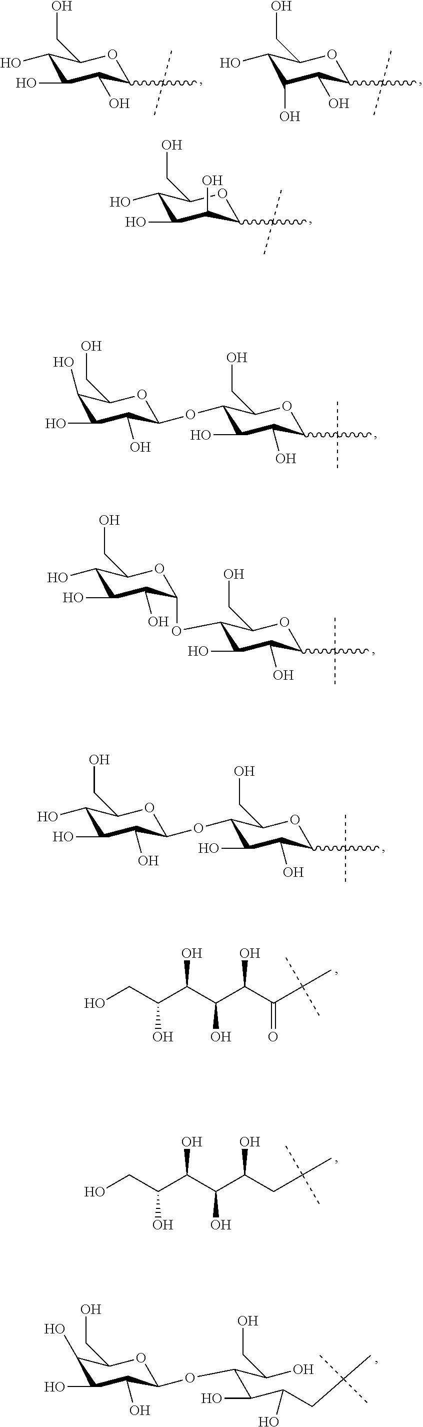

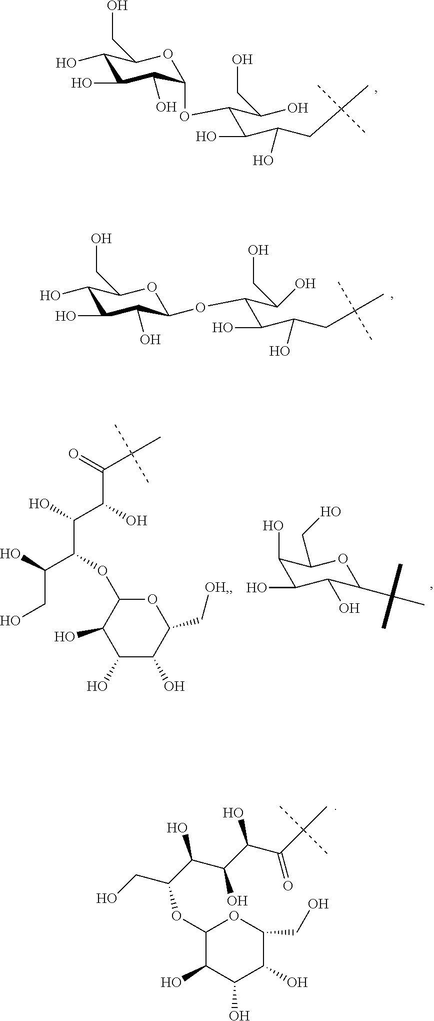

3. The compound as claimed in claim 1,
wherein
R¹ is hydrogen;
R² selected from the group consisting of hydrogen, and a C₆-C₁₈ alkyl;
L is a C₂-C₆ alkyl;
X is NH, or O;
Y is selected from the group consisting of

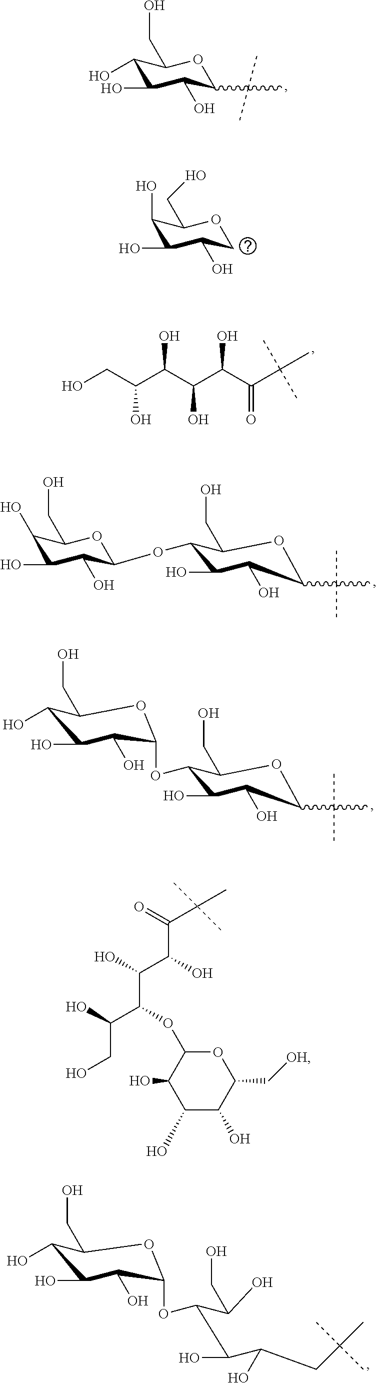

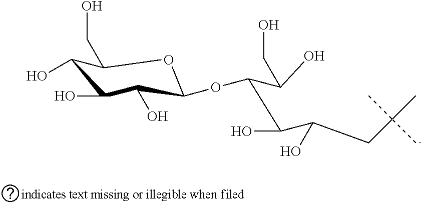

4. A compound of formula (I) as claimed in claims for its stereoisomers, prodrugs and pharmaceutically acceptable salts thereof, which is selected from a group consisting of:

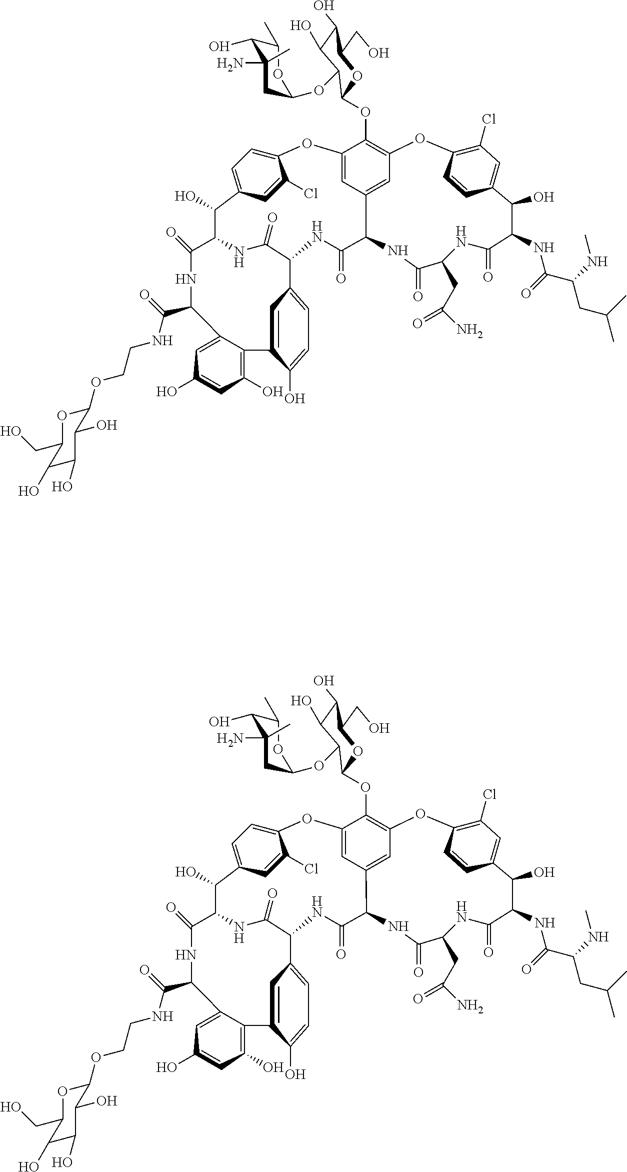

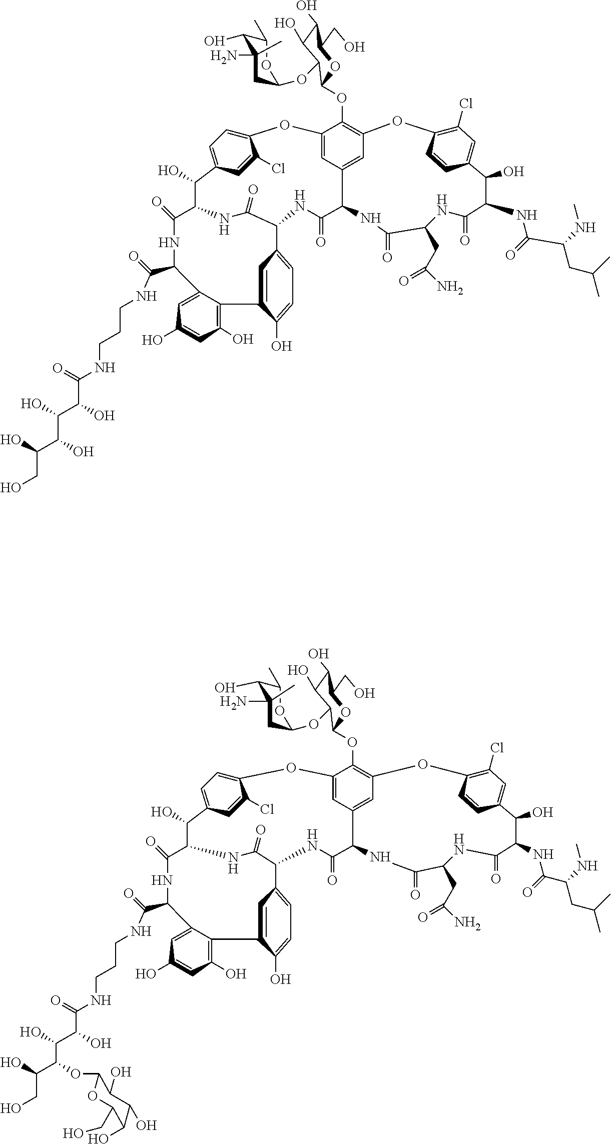

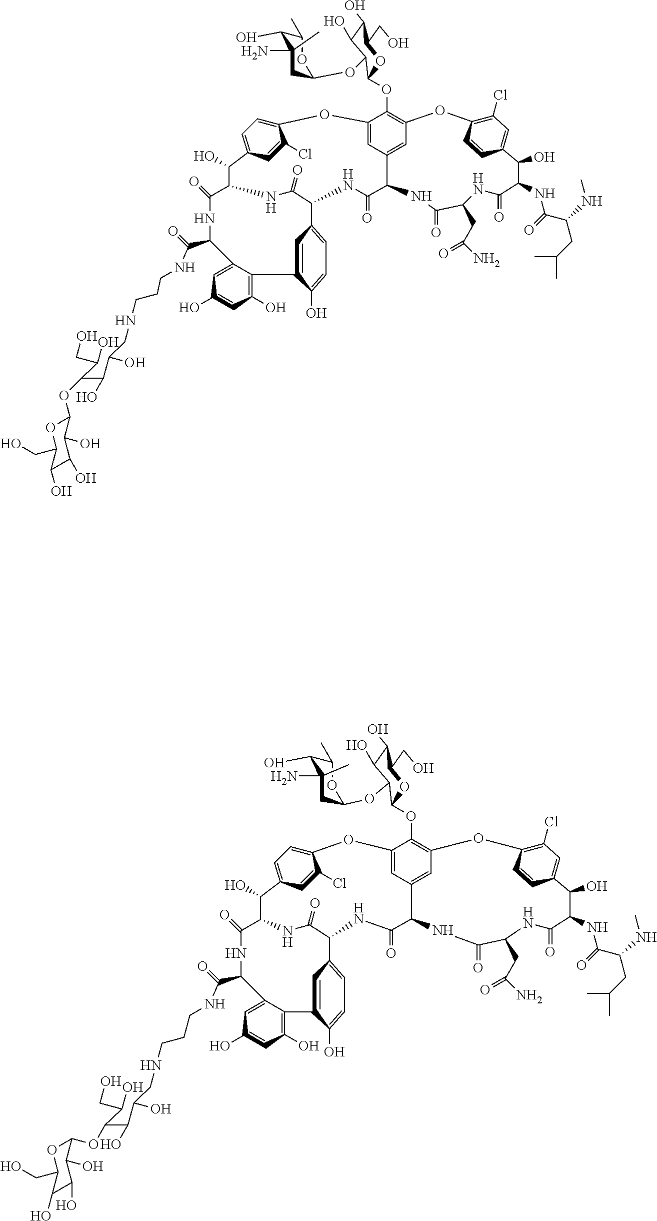

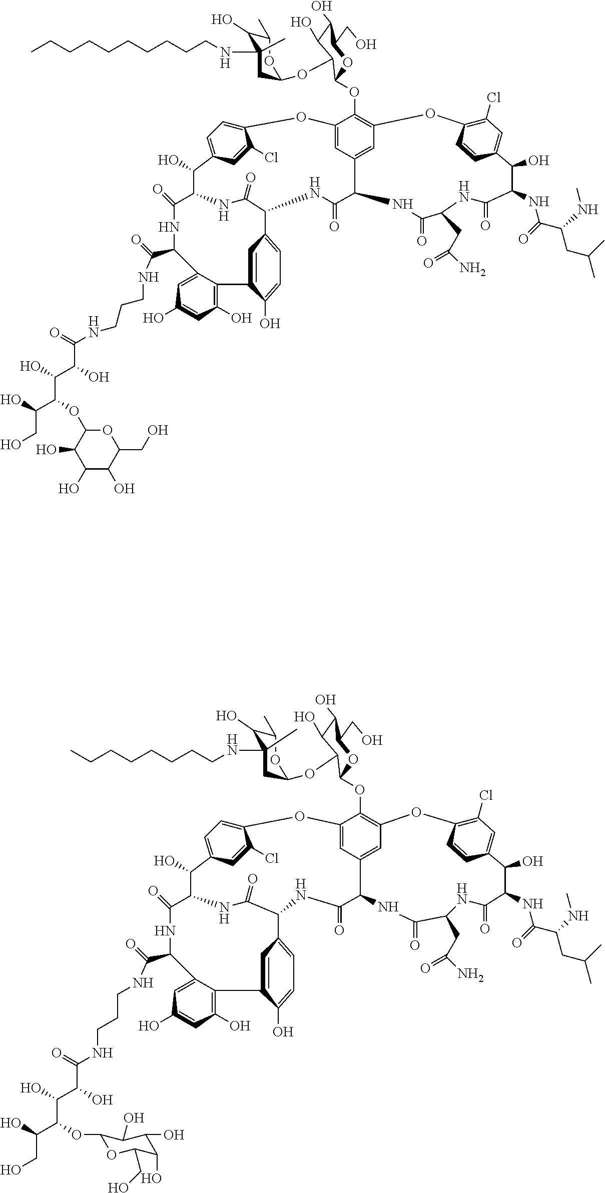

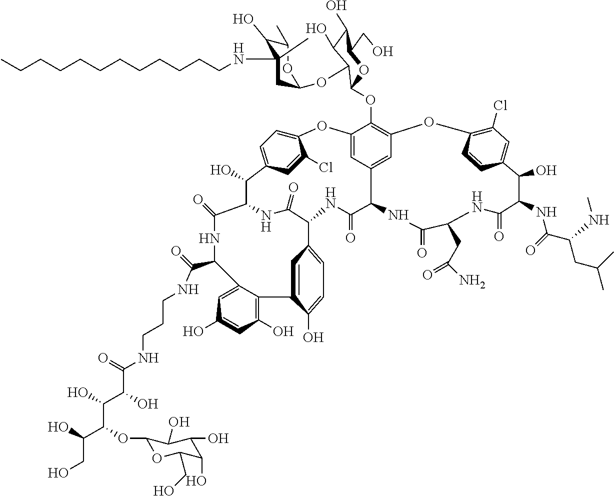

5. A compound as claimed in claim 1 or its stereoisomers, prodrugs and pharmaceutically acceptable salts thereof for use as a medicament.

6. A compound as claimed in claim 1 or its stereoisomers, prodrugs and pharmaceutically acceptable salts thereof for use in treatment of a bacterial infection.

7. The compound as claimed in claim 6 or its stereoisomers, prodrugs and pharmaceutically acceptable salts thereof for use in the treatment of diseases caused by gram positive bacteria.

8. The compound as claimed in claim 6 or its stereoisomers, prodrugs and pharmaceutically acceptable salts thereof for use in treatment of a bacterial infection, wherein the bacterium comprises a vancomycin-resistant bacterium or a methicillin-resistant bacterium.

9. The compound of as claimed in claim 8 or its stereoisomers, prodrugs and the pharmaceutically acceptable salts thereof for use in treatment of a bacterial infection, wherein the bacterium comprises a vancomycin-resistant Staphylococcus aureus, a vancomycin-resistant Enterococcus faecium or a methicillin-resistant Staphylococcus aureus.

10. A pharmaceutical composition comprising a compound of formula (I) or a pharmaceutically acceptable salt thereof of as claimed in claim 1 together with a pharmaceutically acceptable carrier, optionally in combination with one or more other pharmaceutical compositions.

11. A method of preparing the pharmaceutical composition as claimed in claim 10.

12. A method of killing a bacterial cell, the method comprising contacting the cell with a compound as claimed in claim 1, or its stereoisomers, prodrugs and pharmaceutically acceptable salts thereof, in an amount sufficient to kill the bacterial cell.

13. The method as claimed in claim 12, wherein the bacterial cell is selected from the group consisting of enterococci, staphylococci, and streptococci.

14. A method for treatment of bacterial infection in a subject comprising: administering to the subject an effective amount of the compound of claim 1 or its stereoisomers, prodrugs and pharmaceutically acceptable salts thereof.

15. The method of claim 14 wherein the bacterial infection is caused by a gram-positive bacterium.

16. The method of claim 14, wherein the bacterial infection comprises an infection caused by a drug-resistant bacterium.

17. The method of claim 16, wherein the drug-resistant bacterium is a vancomycin-resistant bacterium or a methicillin-resistant bacterium.

18. The method of claim 16, wherein the bacterium comprises a vancomycin-resistant Staphylococcus aureus, a vancomycin-resistant Enterococcus faecium or a methicillin-resistant Staphylococcus aureus.

19. An article comprising: a composition comprising the compound of claim 1 or its stereoisomers, prodrugs and pharmaceutically acceptable salts thereof.

20. An article comprising a substrate, wherein the substrate is coated with or impregnated with the composition comprising the compound of claim 1 or its stereoisomers, prodrugs and pharmaceutically acceptable salts thereof.

21. A process of preparation of compound of formula (I) as claimed in claim 1 or stereoisomers, prodrugs and pharmaceutically acceptable salts thereof.
